(19) United States
(12) Patent Application Publication
(10) Pub. No.: US 2024/0131359 A1
(43) Pub. Date: Apr. 25, 2024

(54) PHANTOM AND ASSOCIATED SYSTEMS AND METHODS FOR STEREOTACTIC BODY RADIATION THERAPY (SBRT)

(71) Applicant: Hackensack Meridian Health, Inc., Edison, NJ (US)

(72) Inventors: Alois Ndlovu, Edison, NJ (US); John Napoli, Edison, NJ (US); Roland F. Teboh, Edison, NJ (US); Brett Lewis, Edison, NJ (US); Alexander Kapulsky, Edison, NJ (US); Joseph Barbiere, Edison, NJ (US)

(73) Assignee: Hackensack Meridian Health, Inc., Edison, NJ (US)

(21) Appl. No.: 18/484,324

(22) Filed: Oct. 10, 2023

Related U.S. Application Data
(60) Provisional application No. 63/415,591, filed on Oct. 12, 2022.

(51) Int. Cl. A61N 5/10; G16H 20/40; G16H 40/40

(52) U.S. Cl. CPC A61N 5/1039; A61N 5/1045; G16H 20/40; G16H 40/40; A61N 2005/1076

(57) ABSTRACT

A phantom for quality assurance for stereotactic body radiation therapy, systems and methods are provided. In some embodiments, a phantom has a body including a peripheral ring potion defining a ring shaped peripheral film holder and a center portion defining a central axis film holder for receiving a radiochromic film.

### RELATED APPLICATION

The present application claims benefit under 35 U.S.C. § 119(e) of U.S. Provisional Patent Application No. 63/415,291, filed Oct. 12, 2022, the content of which is incorporated by reference herein in its entirety.

### BACKGROUND OF THE INVENTION

Conventional radiation typically is delivered in relatively small doses each day over several weeks, which can delay or interfere with chemotherapy. Intensity modulated radiation therapy (IMRT) is an advanced type of radiation therapy used to treat cancer and noncancerous tumors which uses many precisely focused photon or proton radiation beams of varying intensities to precisely irradiate a tumor. The radiation intensity of each beam is controlled, and the beam shape changes throughout each treatment. The goal of IMRT is to conform the radiation dose to the target and to avoid or reduce exposure of healthy tissue to limit the side effects of treatment. IMRT typically delivers a complete course of radiation in 7 to 9 weeks.

IMRT delivery techniques can be divided into two broad categories, fixed and moving gantry. Fixed-gantry IMRT delivery employs step-and-shoot (or segmental) sliding window (dynamic) or compensator-based methods. [Moften, M. et al. Tolerance limits and methodologies for IMRT measurement-based verification QA: recommendations of AAPM Task Group No. 218.” Medical Physics (2018) 45 (4): e53-e83]. Moving gantry IMRT delivery includes spiral or sequential tomotherapy [Id., citing Mackie, T R et al. Med. Phys. (19993) 20: 1709; Low, D A et al. Intl J. Radiat. Oncol. Biol. Phys. (1998) 42: 681-92] and volumetric modulated arc therapy (VMAT). The tomotherapy technique uses specialized binary multileaf collimators (MLCs) with only open and closed positions. VMAT requires dynamic MLC delivery along with continuously variable or quantized dose rate and gantry motion. [Id., citing Ling, C C et al. Intl J. Radiat. Oncol. Biol. Physi. (1998) 42: 681-92; Kaurin, D G et al. Appl. Clin. Med. Phys. (2012 13: 3725; Otto, S. Med. Phys. (2008) 35: 310-17].

IMRT dose distributions are typically much more heterogeneous than those of three-dimensional (3D) plans, employing complex fields that incorporate different degrees of modulation. [Moften, M. et al. Tolerance limits and methodologies for IMRT measurement-based verification QA: recommendations of AAPM Task Group No. 218.” Medical Physics (2018) 45 (4): e53-e83]. Since the inception of IMRT, procedures for the delivery system and patient-specific IMRT plan quality assurance (QA) have emerged [Id, citing Low, D A et al. Intl J. Radiat. Oncol. Biol. Phys. (1998) 42: 681-92] based on measurement and calculation techniques, including independent monitor unit (MU) calculations for IMRT. [Id., citing Xing, L. et al. Monitor unit calculation for an intensity modulated photon field by a simple scatter-summation algorithm. Phys. Med. Biol. (2000) 45: N1]. IMRT QA verification is a process employed to check the accuracy of IMRT plan dose calculations and to detect clinically relevant errors in the radiation delivery, thereby ensuring the safety of patients and fidelity of treatment.

An early American Association of Physicists in Medicine (AAPM) report on IMRT clinical implementation described delivery systems and pretreatment QA. [Id., citing Ezzell, G A et al. Med. Phys. (2003) 30: 2089-2115]. In 2009, additional details concerning IMRT commissioning were addressed including tests and sample accuracy results for different IMRT planning and delivery systems. [Id., citing Ezzell, G A et al. Med. Phys. (2009) 36: 5359-73]. In 2011, strengths and weaknesses of different dosimetric techniques and the acquisition of accurate data for commissioning patient-specific measurements were addressed. [Id., citing Low, D A et al. Med. Phys. (2011) 38: 1313-38]. A comprehensive White Paper on safety considerations in IMRT was also published, which specified that pretreatment validations were necessary [Id., citing Moran, J M et al. Med. Phys. (2011) 38: 5067] for patient safety, but the goal of the White Paper was not to explicitly address how that validation should be done. Other possibilities besides measurements have been published, including independent computer calculations, check-sum approaches, and log file analysis [Id., citing Xing, L. et al. Phys. Med. Biol. (2000) 45: N1; Pawlicki, T. et al. Radiother. Oncol. (2008) 89: 330-37; Fan, J. et al. Phys. Med. Biol. (2006) 51: 2503-14; Leal, A. et al. Intl J. Radiat. Oncol. Biol. Phys. (2003) 56: 58-68; Agnew, A. et al. Phys Med Biol. (2014) 59: N49-N63; Rangaraj, D. et al. Pract. Radiol. Oncol. (2013) 3: 80-90; Stell, A M et al. Med. Phys. (2004) 31: 1593-1602]. Several professional organizations (AAPM, American College of Radiology (ACR), American Society for Radiation Oncology (ASTRO) [Id, citing Ezzell, G A et al. Med Phys. (2003) 30: 2089-2115; Ezzell, G A et al. Med. Phys. (2009) 36: 5359-73; Moran, J M et al. Med. Phys. (2011) 38: 5067; Hartford, A C et al. Am. J. Clin. Oncol. (2012) 35: 612-17] have strongly recommended patient-specific IMRT QA be employed as part of the clinical IMRT process. Measurement-based patient-specific IMRT QA methods are widely used and are the core element of most IMRT QA programs. In many centers, a QA measurement is routinely performed after a patient's IMRT plan is created and approved by the radiation oncologist. The treatment plan consisting of MLC leaf sequence files (or compensators) as a function of gantry angle and MUs from the patient's plan is computed on a homogeneous phantom to calculate dose in the QA measurement geometry. The physical phantom is irradiated under the same conditions to measure the dose. The calculations and measurements are compared and approved or rejected using the institution's criteria for agreement. If the agreement is deemed acceptable, then one infers that the delivered patient plan will be accurate within clinically acceptable tolerances. This phantom plan does not check the algorithm's management of heterogeneities, segmentation errors, or patient positioning errors. The details of methods used to evaluate the agreement between measured and calculated dose distributions however are often poorly understood by the medical physicists.

The acceptance criteria for patient-specific IMRT-QA are difficult to establish because of large variations among IMRT planning systems, delivery systems, and measurement tools [Id., citing Ibbott, G S, et al. Intl J. Radiat. Oncol. Biol. Phys. (2008) 71: S71-S75; Palta, J R et al. Intl J. Radiat. Oncol. Biol. Phys. (2004) 59: 1257-59; Palta, J R et al. In J R Palta, T R Mackie, eds. Intensity-Modulated Radiation Therapy: The State of Art. Madison: Medical Physics Publishing (2003) 593-612; Das, I J et al. J. Natl Cancer Inst. (2008) 100: 300-7]. There are many sources of errors in IMRT planning and delivery. In terms of treatment planning, the error sources can include modeling of the MLC leaf ends, MLC tongue-and-groove effects, leaf/collimator transmission, collimators/MLC penumbra, compensator systems (scattering, beam hardening, alignment), output factors for small field sizes, head backscatter, and off-axis profiles. They can also include a selection of the dose calculation grid size and the use and modeling of heterogeneity corrections. Accurate IMRT treatment planning systems (TPS) beam modeling is essential to reduce the uncertainties associated with the TPS planning process and consequently ensure good agreement between calculations and measurements when performing patient-specific verification QA [Id., citing LoSasso, T. et al., Med. Phys. (2001) 28: 2209-19; Alber, M. et al. ESTRO booklet (2008)].

Spatial and dosimetric delivery system uncertainties also affect IMRT dose distribution delivery accuracy. These uncertainties include: MLC leaf position errors (random and systematic), MLC leaf speed acceleration/deceleration, gantry rotational stability, table motion stability, and beam stability (flatness, symmetry, output, dose rate, segments with low MUs). In addition, differences and limitations in the design of the MLC and accelerators, including the treatment head design and age of the accelerator/equipment can have an impact on the accuracy of IMRT delivery techniques. [Id., citing LoSasso, T. et al., Med. Phys. (2001) 28: 2209-19; Alber, M. et al. ESTRO booklet (2008)].

Another source of uncertainty when using measurement-based patient-specific IMRT QA programs is the measurement and analysis tools used to interpret the QA results [Id., citing Childress, N L et al. Med. Phys. (2005) 32: 153-62; Chuang, C F et al. Med. Phys. (2002) 29: 1109-15; Godart, J. et al. Phys. Med. Biol. (2011) 56: 5029-43; Han, Z. et al. Med. Phys. (2010) 37: 3704-14; Low, D A and Dempsey, J F. Med. Phys. (2003) 30: 2455-64]. These software tools have several parameters that must be chosen to perform the analysis and the results can vary significantly depending on those choices (for example, the selection of whether to use global or local dose normalization to compare measured and calculated dose distributions).

Stereotactic body radiation therapy (SBRT) is a cancer treatment that delivers extremely precise, very intense doses of radiation to cancer cells while minimizing damage to healthy tissue. SBRT involves the use of sophisticated three-dimensional image guidance that pinpoints the exact three-dimensional location of a tumor so that the radiation can be more precisely delivered to cancer cells while minimizing damage to surrounding healthy tissue. Specialized equipment focuses multiple beams of radiation and/or a beam spread over a large angular range on the tumor location. Each individual beam or the beam spread over the large angular range has a relatively smaller effect on the healthy tissue it passes through in comparison to the effect of the large targeted dose of radiation delivered to the tumor location where the beam or beams focus and intersect. SBRT usually can be given in five or fewer daily sessions and requires no anesthesia. For example, SBRT is used to treat tumors in the lungs, spine, liver, neck, lymph node or other soft tissue. SBRT is delivered through linear accelerators, which form beams of fast-moving subatomic particles.

Modern radiotherapy planning systems provide a set of computerized tools that allow the radiation oncologist, medical physicist, and treatment planner to create and visualize radiotherapy treatments, given the imaging data available.

Current patient specific QA procedures for relatively small SBRT targets are inadequate. They are usually performed using either a cylindrical or planar phantom embedded with electronic detectors and utilize commercial analysis algorithms. Although efficient, these commercial devices do not provide data in the axial plane commonly most useful for clinical plan evaluation, have relatively coarse spatial resolution for small SBRT targets, provide no positional data relating results to patient anatomy, and cannot be easily altered for customized calculations besides the standard Gamma Index to include for example information about a specific region of interest (ROI). In contrast, commercial film techniques have excellent spatial resolution, but are often limited to a single plane within a 3D dose distribution. Further, film calibration with multiple color channels is not standardized.

The successful use of high-energy external beam radiation for therapeutic purposes depends critically on the spatial distribution of absolute dose within the patient. The primary reason is that the energy deposition itself is three-dimensional (3D) in nature, where particles not only affect the immediate interaction site, but also deposit some of their energy into the surrounding area. Thus, healthy tissue dose tolerance often becomes a limiting factor to treatment success. Precise knowledge and control of the dose distribution allows one to approach this intrinsic limit closely and in a controlled manner, thereby maximizing the radiation's therapeutic effect. The treatment process entails designing a 3D dose distribution through the use of imaging data for the modeling of anatomy and dose deposition. The accuracy of this process needs to be validated through measurement of dose.

Dose Difference/Distance to Agreement, γ Analysis, and Verification Metrics.

Dose distributions are almost always represented as arrays of points, each defined by a location and dose value. The spacing between the points is the spatial resolution of the distribution, and does not need to be the same in all spatial dimensions or locations. The spatial resolution of the dose distribution plays an important role in its display and evaluation. Course dose distribution may require some type of interpolation to display in an easily interpretable form, such as isodose lines or dose color washes. Dose distribution resolution also plays a role in dose distribution comparisons. Some comparison techniques are degraded by coarse resolution, so interpolation is employed.

A common method for comparing dose distributions is to overlay their contours. If the distributions agree exactly, the contours will overlay and if not, they will separate. The separation distance will depend on two factors: the difference in the doses and the local dose gradients. When the gradients are steep, contours move only slightly with changes in dose, so even large dose errors will correspond to small contour separations. Therefore, comparing contours in steep dose gradient regions provides little insight as to the dose differences because it takes very large differences to significantly move the lines. On the other hand, even small dose differences will move isodose lines far in low-dose gradient regions. The only places where contour plots easily provide quantitative information are where isodose lines cross or superimpose. If the isodose lines are the same values, the distributions agree exactly at those locations. If two different isodose lines cross, for example the 50% line from one distribution and the 60% line from the other distribution, the dose difference is known at the crossing point. Otherwise, superimposed isodose contours provide little quantitative information.

The goal of dose comparison is to determine if the reference and evaluated dose distributions agree to within limits that are clinically relevant. The question of clinical relevance involves more than the dose itself; it also involves the dose gradients and dose errors resulting from spatial uncertainties. There is therefore a need to understand both the spatial and dosimetric uncertainties when conducting dose distribution comparisons.

For this, various representative objects or “phantoms” act as a surrogate to the patient anatomy. [Frigo, S. P. (2014). Radiation Therapy Dosimetry Phantoms. In: DeWerd, L., Kissick, M. (eds) The Phantoms of Medical and Health Physics. Biological and Medical Physics, Biomedical Engineering. Springer, New York, NY. https://doi.org/10.1007/978-1-4614-8304-5_2]. A phantom for therapeutic dose measurement must satisfy a number of basic design goals related to materials first and geometry second. For materials, they must be similar to tissue such that measurements can be mapped to dose to tissue; have composition that can be well characterized and readily available; allow for easy traceability to reference standards; be robust to radiation damage; and exhibit reproducible and well-understood response with regard to radiation type and energy. For geometries, they must accommodate delivered beam field sizes and shapes; allow the establishment of 3D locations; be easy to transport, set up, align and take down in an accurate and efficient manner. For phantoms in routine use, the design must be rugged enough to allow repeated handling and use by technical staff.

### SUMMARY OF THE INVENTION

In one aspect, the present disclosure provides a phantom, comprising: a body including a peripheral ring portion defining a central axis of the body and of the phantom and a center portion (e.g., a center cylindrical portion) at least partially encircled by the peripheral ring portion or fully encircled by the peripheral ring portion, the center portion aligned with and extending along the central axis of the body; a peripheral film holder formed in the peripheral ring portion and at least partially encircling or fully encircling the central axis, the peripheral film holder configured to receive at least one peripheral film; and an axial film holder formed in the center portion and oriented perpendicular to the central axis, the axial film holder configured to receive at least one axial film. In some embodiments of the phantom, the peripheral ring portion fully encircles the center portion. In some embodiments, the peripheral film holder fully encircles the central axis. In some embodiments, the phantom is configured to be exposed to radiation such that both the at least one peripheral film and the at least one second axial film are simultaneously exposed to the radiation. In some embodiments, the peripheral ring portion includes an inner ring and an outer ring at least partially encircling the inner ring, at least a portion of the outer ring removably coupled to or configured to be removably coupled with the inner ring. In some embodiments, at least a portion of the outer ring is removable from the inner ring to expose at least about 180° of a film supporting surface of the inner ring. In some embodiments, the peripheral film holder is defined by a radially inward facing surface of the outer ring and a radially outward facing surface of the inner ring. In some embodiments, the peripheral film holder extends at least about 180° about the center axis between the outer ring and the inner ring. In some embodiments, the peripheral film holder extends at least about 270° about the center axis between the outer ring and the inner ring. In some embodiments, the peripheral film holder extends about 360° about the center axis between the outer ring and the inner ring. In some embodiments, the peripheral film holder is configured such that at least one peripheral film positioned in the peripheral film holder would have a front surface or a back surface facing a cylindrically shaped surface of the peripheral film holder with an axis of a cylinder of the cylindrically shaped surface being the central axis of the phantom. In some embodiments, the center portion includes a base and a cap configured to be removably coupled to the base section. In some embodiments, the axial film holder is defined by a surface of the base and a surface of the cap. In some embodiments, the axial film holder is oriented perpendicular to the central axis of the body. In some embodiments, the peripheral film holder configured to receive and hold the at least one peripheral film is configured to hold at least one radiochromic film and axial film holder configured to receive and hold the at least one axial film is configured to hold at least one radiochromic film.

In another aspect, the present disclosure provides a computer-implemented method for determining a density to dose calibration for a radiochromic film to be used with a phantom for evaluating a radiation therapy plan, the method comprising: receiving or accessing data corresponding to a digitized image of a first radiochromic film exposed to radiation while positioned in the phantom according to a calibration plan including multiple different levels of radiation along the first radiochromic film and generating two-dimensional film density data corresponding to the received and accessed data for the first radiochromic film; determining an average film density profile along a direction in the two-dimensional film density data by averaging a plurality of profiles parallel to the direction; determining a plan dose profile corresponding to the direction in the film based on the calibration plan; determining a best fit fourth order polynomial and corresponding coefficients for plan dose as a function of film density for the first radiochromic film based on the determined average film density profile and the determined plan dose profile, thereby determining a density to dose calibration for the first radiochromic film. The method may also include applying the best fit fourth order polynomial as a calibration curve for received or accessed data regarding a second exposed radiochromic film having the same film characteristics as those of the first radiochromic film and used with the phantom for evaluating the radiation therapy plan. In some embodiments, the method further comprises generating the calibration plan based on one or more characteristics of a patient-specific treatment plan. In some embodiments of the method, the first radiochromic film was disposed at a peripheral ring of the phantom that encircled a central axis of the phantom during exposure. In some embodiments of the method, the cylindrical phantom is the phantom as described above.

In another aspect of the present disclosure, a computer-implemented method for determining a gamma index for verification or quality assurance of a radiation therapy patient treatment plan (e.g., an SBRT patient treatment plan) includes: receiving or accessing plan data regarding the radiation therapy patient treatment plan; receiving or accessing exposed film data corresponding to at least one digitized image of at least one radiochromic film exposed to radiation in a cylindrical phantom according to the radiation therapy patient treatment plan; converting density in the at least one digitized film image to film dose to produce at least one two-dimensional film dose map using a calibration curve; receiving information regarding or identifying a geometric region of interest; determining film dose values corresponding to the geometric region of interest from the at least one two-dimensional film dose map; determining plan dose values for the radiation therapy patient treatment plan corresponding to the geometric region of interest; and generating a gamma index specific to the region of interest based, at least in part, on the determined film dose values and determined plan dose values corresponding to the geometric region of interest.

In some embodiments of the method, the at least one film includes a film that was positioned on a cylindrical surface of the phantom during exposure. In some embodiments, the at least one film includes a film that was at a center of the phantom and oriented perpendicular to a central axis of the phantom during exposure. In some embodiments, the at least one film includes at least one peripheral film that was disposed on a cylindrical surface of the phantom during exposure and at least one central film that was disposed at the center of the phantom and was oriented perpendicular to a central axis of the phantom during exposure. In some embodiments, the region of interest includes an area corresponding to a position of the at least one peripheral film. In some embodiments, the received information regarding or identifying the geometric region of interest is received from a user.

In another aspect, the present disclosure provides a computer-implemented method for quality assurance of a radiation therapy patient treatment plan, the method including: receiving data corresponding to a digitized image of a radiochromic film exposed to radiation in a cylindrical phantom according to the radiation therapy patient treatment plan with the radiochromic film disposed at a central axis of the cylindrical phantom and oriented perpendicular to the cylindrical phantom; converting density in the digitized radiochromic film image to film dose to produce a two-dimensional film dose map using a calibration curve; determining a plan dose map corresponding to a plane of the radiochromic film from the radiation therapy patient treatment plan; receiving or determining an isodose curve for a first isodose value for the film dose map; receiving or determining an isodose curve for a first isodose value for the plan dose map; determining a radial distance to the isodose curve for the film dose map and determining a radial distance to the isodose curve for the plan dose map at each of a plurality of orientations; and displaying radial distances to the isodose curves for the film dose map and for the plan dose map at the plurality of orientations in polar coordinates.

In another aspect, the present disclosure provides a computer readable medium storing instructions that when executed by at least one processor performs the computer-implemented method for determining a density to dose calibration for a radiochromic film to be used with a phantom for evaluating a radiation therapy plan; the computer-implemented method for determining a gamma index for verification or quality assurance of a radiation therapy patient treatment plan; and/or the computer-implemented method for quality assurance of a radiation therapy patient treatment plan.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. To assist those of skill in the art in making and using the disclosed phantom and associated systems and methods, reference is made to the accompanying figures, wherein:

FIG. 1A is a perspective view of a phantom in accordance with some embodiments of the present disclosure.

FIG. 1B is a Computerized Tomography (CT) axial view of the phantom depicted in FIG. 1A.

FIG. 1C is a CT coronal view of the phantom depicted in FIG. 1A.

FIG. 2 is a perspective view of a color scanner scanning a peripheral radiochromic film after its exposure to a calibration radiation plan when disposed in the phantom in accordance with some embodiments.

FIG. 3 is a uniformity correction plan determined by an exemplary system in accordance with some embodiments.

FIG. 4 is a top view of an exposed radiochromic film used for determination of a uniformity correction plan in accordance with some embodiments.

FIG. 5A is a graph of a plan dose converted to density and measured film density y-axis) with respect to distance (x-axis), where 0.5 mm=1 pixel (gray dots-film, black line-plan), with agreement within 2% in accordance with some embodiments.

FIG. 5B is a graph of a ratio of film density to plan density (y-axis) with respect to distance (x-axis), where 0.5 mm=1 pixel (gray dots-film, black line-plan), having a slope of 0.004 indicating uniform scanner response in a direction for a scan region used in an example in accordance with some embodiments.

FIG. 6 is a phantom plan generated with multiple static fields to evaluate a phantom in accordance with some embodiments.

FIG. 7 is a dose calibration plan generated with an exemplary system in accordance with some embodiments.

FIG. 8 is a top view of a film exposed to radiation according to the dose calibration plan of FIG. 7 in accordance with some embodiments.

FIG. 9 is a graph of measured film dose as compared to a plan dose (y-axis) versus distance (0.5 mm=1 pixel, x-axis) in accordance with some embodiments.

FIG. 10 is a graph of a fourth-degree polynomial order fit to a calibration curve for dose versus channel intensity for the graph of FIG. 9 in accordance with some embodiments.

FIG. 11 is a representative stereotactic body radiation therapy (SBRT) plan generated by an exemplary volumetric modulated arc therapy (VMAT) SBRT lung patient plan with two 180-degree arcs created with the planning target volume (PTV) near the brachial plexus.

FIG. 12 is a view of a verification plan including a peripheral portion corresponding to FIG. 11 that was created on the phantom with doses extracted in the periphery encompassing the entire treatment field in accordance with some embodiments.

FIG. 13 is a central region view of the verification plan created on the phantom with doses extracted in the clinically significant axial plane where the brachial plexus is closest to exceeding tolerance in accordance with some embodiments.

FIG. 14 is a top view of an exposed peripheral film in accordance with some embodiments.

FIG. 15 is a graph of a profile plot of the measured film plan in FIG. 14 as compared to a corresponding plan profile (Red:=Measured film; Black=Plan) in accordance with some embodiments.

FIG. 16 is a scatter plot for parameter verification of film dose (y-axis) vs plan dose (x-axis) (black) showing superb agreement validating the procedure using a linear fit slope (Red: Best linear fit slope=0.997) in accordance with some embodiments.

FIG. 17 is a scatter plot of dose (y-axis) vs. film density (x-axis) for a calibration curve showing good agreement with patient data for validation (Red: Peripheral film; Black: calibration curve) in accordance with some embodiments.

FIG. 18A is a polar plot showing measured film (red) and plan (black) isodose curves for the same isodose at the center axis film for various angles in accordance with some embodiments.

FIG. 18B shows error (mm (y-axis) vs. angle (x-axis) for the film measured plan of FIG. 18A. The film is misregistered to the plan and results in large variations in radial distances exceeding 3 mm.

FIG. 19A is a polar plot showing a measured film (red) and planned (black) isodose curves for the same isodose at the center axis film for various angles in accordance with some embodiments.

FIG. 19B is a plot of error (mm, y-axis) vs. angle (x-axis) showing good matching at a region of interest (ROI) tolerance dose DH<2 mm for the measured film vs. plan of FIG. 19A in accordance with some embodiments.

FIG. 20A is a polar plot of measured scanned film (red) and plan (black) isodose curves for the same isodose at the center axis film for various angles in accordance with some embodiments.

FIG. 20B is a plot of error (mm) (y-axis) vs. angle (x-axis) for the measured film of FIG. 20A showing an excellent result for target HD=0.5 mm in accordance with some embodiments.

FIG. 21 schematically depicts a network for implementing some methods or in which or with which some phantoms and systems may be employed, in accordance with some embodiments; and

FIG. 22 schematically depicts a computing system for implementing some methods in which or with which some phantoms and systems may be employed, in accordance with some embodiments.

### DETAILED DESCRIPTION OF THE INVENTION

Glossary

The term “absorbed dose” as used herein refers to the amount of energy deposited per unit mass. The units for absorbed dose are gray (Gy, international unit) and rad (rad, U.S. unit).

The term “action limits” as used herein refers to the amount that quality measures are allowed to deviate without risking harm to the patient [Moften, M. et al. Tolerance limits and methodologies for IMRT measurement-based verification QA: recommendations of AAPM Task Group NO. 218.” Medical Physics (2018) 45 (4): e53-e83, citing 35] as well as defining limit values for when clinical action is required.

Anatomical Terms. When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is a Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, and lateral structures are further from the midsagittal plane. Structures in the midline of the body are median. For example, the tip of a human subject's nose is in the median line.

Ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

The term “collimator” as used herein refers to a device that limits the radiation output of an X-ray source. A multileaf collimator (MLC) consists of shutters, slats, or vanes (generically referred to as leaves) positioned between the x-ray source and the target in order to define field shape by subtracting portions of the primary beam.

The term “distance to agreement” or “DTA” refers to the distance between common features of the reference distribution and the evaluated distribution, particularly in steep dose gradient regions. For a point in the reference distribution, the DTA is the closest location in the evaluated dose distribution with the same dose as the point in the reference distribution.

Dose Difference Test. The dose difference at location (r) is the numerical difference δ between the evaluated dose (Dₑ(\vec{r})) and the reference dose (Dᵣ(\vec{r})) at that location.

The term “gamma index” or “γ” is a metric for the verification of complex radiotherapy deliveries such as IMTR and volumetric modulated arc radiotherapy (VMAT).

The term “helical tomotherapy” as used herein refers to a form of IMRT that delivers radiation in a special way. For this treatment, the radiation machine delivers many small beams of radiation at the tumor from different angles around the body.

The term “image guided radiation therapy” or “IGRT” as used herein refers to a form of 3D-CRT where imaging scans (like a CT scan) are done before each treatment. This allows the radiation oncologist to adjust the position of the patient or re-focus the radiation as needed to be sure that the radiation beams are focused on the tumor exactly and that exposure to normal tissues is limited.

The term “intensity modulated radiation therapy” or “IMRT” refers to a radiation therapy like 3D-CRT, but it also changes the strength of some of the beams in certain areas. This allows stronger doses to get to certain parts of the tumor and helps lessen damage to nearby normal body tissues.

The term “isodose curve” or isodose contour” as used herein is a line of constant absorbed dose. The line is in a plane and for single radiation beams, its value is usually related by a simple percentage value to the peak absorbed dose on the beam axis. Isodose curves or isodose contours refer to lines passing through points of equal dose. A set of isodose curves, usually drawn for regular intervals of percentage depth dose, is known as an “isodose chart”. An isodose chart for a given beam consists of a family of isodose curves usually drawn at equal increments of percent depth dose, representing the variation in dose as a function of depth and transverse distance from the central axis. [Isodose charts for single fields. Reports of the Intl Commn on radiation units and measurements. (1973); os-12(2): 16-20].

The term “linear energy transfer (LET)” is used to indicate the average amount of energy that is lost per unit path-length as a charged particle travels through a given material. The LET for electrons is traditionally expressed in units of MeV/cm, or, when divided by the mass density, in units of MeV-cm²/g. Higher LET radiations (particles: alpha particles, protons, and neutrons) produce greater damage in a biologic system than lower LET radiations (electrons, gamma rays, x-rays).

The term “medical linear accelerator” or “LINAC” as used herein refers to a device that most commonly used for external beam radiation treatments for patients with cancer. It delivers high-energy x-rays or electrons to the region of the patient's tumor. These treatments can be designed in such a way that they destroy the cancer cells while sparing the surrounding normal tissue. The linear accelerator uses microwave technology (similar to that used for radar) to accelerate electrons in a part of the accelerator called the “wave guide,” then allows these electrons to collide with a heavy metal target to produce high-energy x-rays. These high energy x-rays are shaped as they exit the machine to conform to the shape of the patient's tumor and the customized beam is directed to the patient's tumor. The beam is usually shaped by a multileaf collimator that is incorporated into the head of the machine. The patient lies on a moveable treatment couch and lasers may be used to make sure the patient is in the proper position. The treatment couch can move in many directions including up, down, right, left, in and out. The beam comes out of a part of the accelerator called a gantry, which can be rotated around the patient. Radiation can be delivered to the tumor from many angles by rotating the gantry and moving the treatment couch.

The term “monitor unit” or “MU” as used herein refers to a measure of radiation “beam-on” time used for medical linear accelerators. By convention, one monitor unit equals 1 cGy of absorbed dose in water under specific calibration conditions for the medical linear accelerator (LINAC). Holmes, T., Ma, C., Wang, L. (2013). Monitor Unit. In: Brady, L. W., Yaeger, T. E. (eds) Encyclopedia of Radiation Oncology. Springer, Berlin, Heidelberg. https://doi.org/10.1007/978-3-540-85516-3_351

The term “phantom” as used herein refers to a specialized object or device that is used as a “stand-in” for human tissue to calibrate and characterize delivered external radiation beams; and validation of numerical dose modeling and design through treatment planning so that delivered and planned dose are as close as possible. Frigo, S. P. (2014). Radiation Therapy Dosimetry Phantoms. Chapter 2, In: DeWerd, L., Kissick, M. (eds) The Phantoms of Medical and Health Physics. Biological and Medical Physics, Biomedical Engineering. Springer, New York, NY. http s ://doi.org/10.1007/978-1-4614-8304-5_2

The abbreviation “QA” as used herein stands for quality assurance.

The term “radiation therapy dosimetry phantom” as used herein refers to a device used in the verification of modeled/planned dose distributions.

The term “radiation treatment planning and delivery ” refers to a process that relies on advanced imaging, computing technology, and expertise from the medical team to generate a radiation treatment plan that will deliver a therapeutic dose of radiation to a tumor while sparing nearby normal tissue.

The term “reference distribution” as used herein refers to the distribution against which an evaluated distribution is being compared.

The term “spatial interpolation” as used herein refers to a process of using points with known values to estimate values at other unknown points.

The term “stereotactic radiosurgery” as used herein refers to a type of radiation treatment used for brain tumors and other tumors inside the head that gives a large dose of radiation from many different angles to a small tumor area, usually in one session. There is no incision or cutting involved. Treatment outside the brain is called stereotactic body radiation therapy (SBRT). SBRT may be used for certain lung, spine, and liver tumors.

The term “three dimensional conformal radiation therapy” or 3D-CRT″ as used herein refers to an external radiation therapy that delivers radiation beams from different directions designed to match the shape of the tumor. This helps to reduce radiation damage to normal tissues and better kill the cancer by focusing the radiation dose on the tumor's exact shape and size.

The term “tolerance limits” as used herein refers to the boundaries within which a process is considered to be operating normally, that is, subject to only random errors. Results outside of the tolerance limits (or trends moving rapidly toward these boundaries) provide an indication that a system is deviating from normal operation.

The term “volumetric modulated arc therapy” or “VMAT” as used herein refers to a form of IMRT where the dose of radiation is applied to the tumor by continuous 360° rotation of the treatment unit. It uses high energy X-rays in the Mega Voltage (MV) generated by a medical linear accelerator. Very small beams with varying intensities are aimed at a tumor and then rotated 360 degrees around the patient. This results in attacking the target in a complete three-dimensional manner.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates, which may need to be independently confirmed.

### Embodiments

The present disclosure describes a phantom (e.g., a cylindrical phantom) for simultaneous acquisition of dose at the phantom surface (e.g., the peripheral surface) encompassing the entire treatment geometry and cumulative axial dose in a preselected clinically relevant plane in accordance with some embodiments. In some embodiments, the phantom includes a body that defines a peripheral film holder encircling, substantially encircling, or at least partially a central axis of the body that is configured to receive and hold at least one peripheral radiochromic film, and that defines a central axial film holder extending perpendicular to the central axis that is configured to receive and hold at least one axial radiochromic film. The use of radiochromic film enables fine resolution dose measurement. Methods described herein enable accurate dose calibration with verification, scanner correction, and versatile analysis tools such as Gamma Index in specific geometrical regions of interest in accordance with some embodiments.

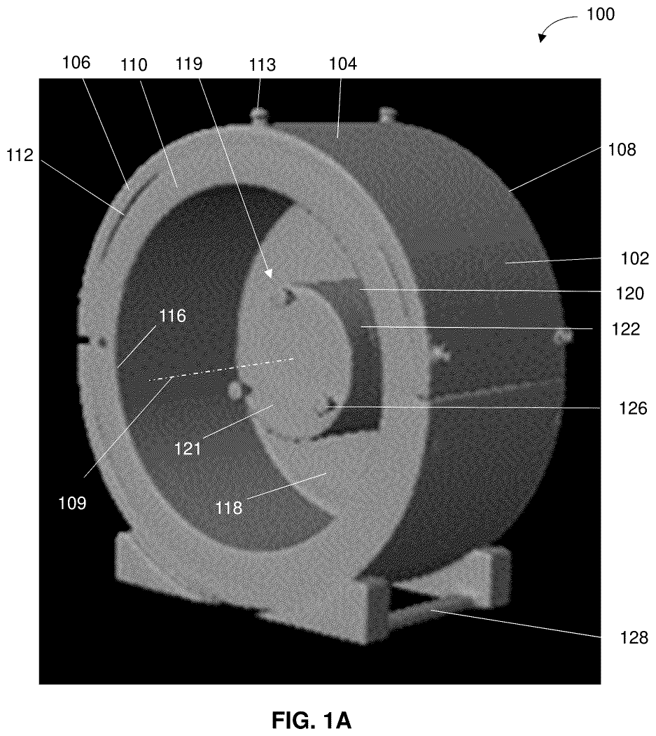

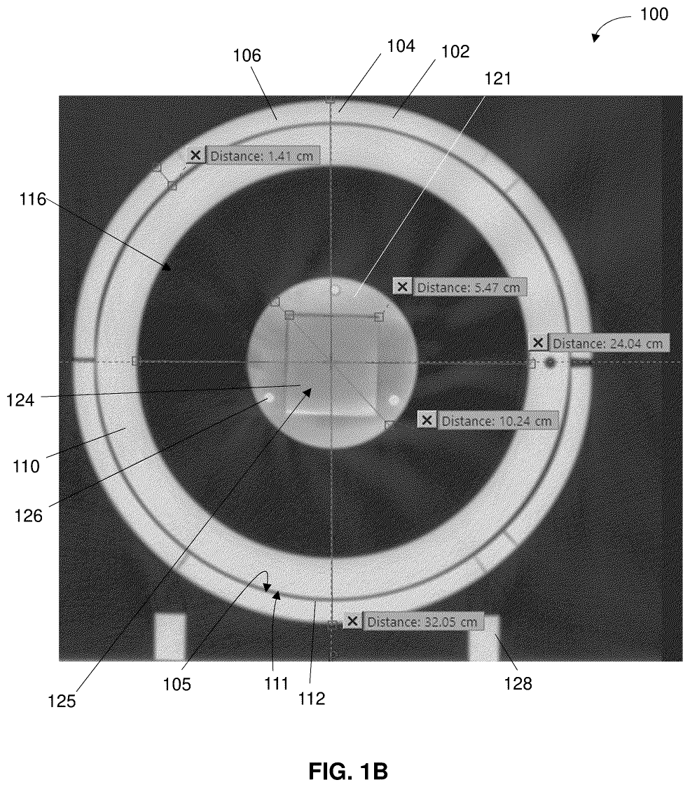

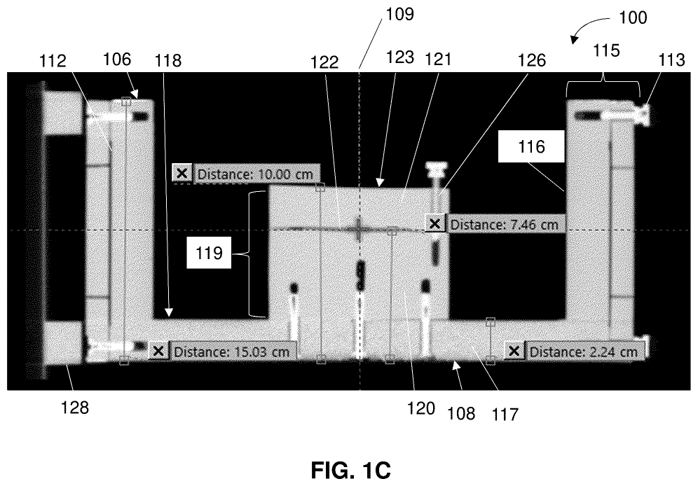

In accordance with embodiments of the present disclosure, an exemplary phantom for use with SBRT is provided. FIGS. 1A-1C are perspective, axial, and cross-sectional views of a phantom 100 in accordance with some embodiments of the present disclosure. In some embodiments, the phantom 100 may include or be fabricated of a polymer material or a plastic material.

In some embodiments, the phantom may include or be fabricated from a material with a density about that of water (e.g., about 1.00 gm/cm³). In some embodiments, the phantom may include or be fabricated from a plastic material having a density in a range of about, e.g., 0.9 to 1.1 gm/cm³ inclusive, 0.91 to 1.1 gm/cm³ inclusive, 0.92 to 1.1 gm/cm³ inclusive, 0.93 to 1.1 gm/cm³ inclusive, 0.94 to 1.1 gm/cm³ inclusive, 0.95 to 1.1 gm/cm³ inclusive, 0.96 to 1.1 gm/cm³ inclusive, 0.97 to 1.1 gm/cm³ inclusive, 0.98 to 1.1 gm/cm³ inclusive, 0.99 to 1.1 gm/cm³ inclusive, 1.0 to 1.1 gm/cm³ inclusive, 0.9 to 1.0 gm/cm³ inclusive, 0.9 to 0.99 gm/cm³ inclusive, 0.9 to 0.98 gm/cm³ inclusive, 0.9 to 0.97 gm/cm³ inclusive, 0.9 to 0.96 gm/cm³ inclusive, 0.9 to 0.95 gm/cm³ inclusive, 0.9 to 0.94 gm/cm³ inclusive, 0.9 to 0.93 gm/cm³ inclusive, 0.9 to 0.92 gm/cm³ inclusive, 0.9 to 0.91 gm/cm³ inclusive, 0.9 gm/cm³, 0.91 gm/cm³, 0.92 gm/cm³, 0.93 gm/cm³, 0.94 gm/cm³, 0.95 gm/cm³, 0.96 gm/cm³, 0.97 gm/cm ³, 0.98 gm/cm³, 0.99 gm/cm³, 1.0 gm/cm³, 1.1 gm/cm³, or the like.

The phantom 100 includes a substantially cylindrical body 102 having a central axis 109 and including an outer ring 104 (e.g., an outer cylinder portion) that defines the outer surface of the phantom 100. In some embodiments, the outer ring 104 generally defines a uniform diameter between a front surface 106 and an opposing rear surface 108 of the body 102.

The phantom 100 also includes an inner ring 110 (e.g., an inner cylinder portion). When the phantom 100 is assembled, the inner ring 110 is encircled by the outer ring 104. At least a portion of the outer ring 104 is spaced from the inner ring by a radial gap or space 112. For example, at least a portion of a radially inward facing surface 105 of the outer ring 104 is spaced from at least a portion of a radially outward facing surface 111 of the inner ring 110 forming the gap or space 112 (e.g., a film slot or opening). This space 112 encircles the inner ring 110 and is configured to receive at least one radiochromic film. This space 112 may be referred to as a film slot 112, or a peripheral film slot, or a peripheral film holder herein. As used herein, the term “film holder” refers to a slot, recess, space, or gap defined by the phantom configured to hold a film. As used herein, a radiochromic film held in the space 112 may be referred to as a peripheral film. In some embodiments, at least a portion of the outer ring 104 is configured to be coupled to the inner ring 110 and uncoupled from the inner ring 110 for the placement or insertion of the at least one radiochromic film. In some embodiments, the inner ring 110 and outer ring 104 are configured to receive fasteners 113 radially spaced from each other along the outer surface of the outer ring 104 to selectively secure the outer ring 104 to the inner ring 110. In some embodiments, the axial length of the outer ring 104 as measured between the front and rear surfaces 106, 108 parallel to a central axis 109 of the phantom 100 is dimensioned greater than the axial length of the inner ring 110. In some embodiments, the axial length of the outer and inner rings 104, 110 as measured between the front and rear surfaces 106, 108 parallel to the central axis 109 is substantially equal such that the front/top surfaces of the rings 104, 110 are substantially aligned.

The combination of the outer ring 104 and the inner ring 110 can be collectively referred to herein as a peripheral ring portion 115 that encircles the central axis 109 of the body 102. In some embodiments, the peripheral ring portion 115 defines an opening 116 at the front surface 106 of the body 102. In some embodiments, the rear surface 108 of the body 102 is closed. In some embodiments, the rear surface 102 of the body 108 is at least partially formed by a support disk portion 117, where the inner ring 110 is coupled to or integral with the support disk portion 117. In some embodiments, the inner ring 110 extends from a front surface 118 of the support disk portion 117.

In some embodiments, the phantom 100 also includes a support or stand 128 on which the body 102 sits or rests in use. In some embodiments, the body 102 may be releasably coupled to or releasably affixed to the support or stand 128.

The phantom 100 also includes a center portion, which may be a center cylindrical portion 119 extending along the central axis 109 of the body 102. In some embodiments, the center portion may have a noncylindrical shape. In some embodiments, the center cylindrical portion 119 extends along the central axis 109 from the front surface 118 of the support disk portion 117 toward a front of the phantom 100. In some embodiments, the center portion is joined to, affixed to, or integral with the support disk portion 117. In some embodiments, the axial length of the center portion 119 measured from the front surface of the support disk 117 to the front surface 123 of the center portion 119 is dimensioned less than the axial length of the inner ring 110 as measured from the front surface 118 of the support disk 117 to the front surface of the body 102 as measured parallel to the central axis 109.

In some embodiments, a radius of the center cylindrical portion 119 is less than an inner radius of the peripheral ring portion such that an annular ring of open space separates the center cylindrical portion 118 and the peripheral ring portion.

In some embodiments, the center portion 119 includes a base 120 and a cap 121 configured to be releasably coupled, mounted, or secured to the base 120 (e.g., using fasteners 126). The base 120 and cap 121 are configured to hold a radiochromic film therebetween oriented perpendicular to the central axis 109 of the body at a distance along the central axis 109 of the body. In some embodiments, the base 120 and cap 121 are configured to define a space or gap therebetween to hold the film oriented perpendicular to the central axis 109. In some embodiments, at least a portion of a surface of the base 120, or a surface of the cap 121, or of both is recessed to form a space, gap or recess to receive the film. For example, the base 120 may have a front surface defining a recess configured to receive one or more axial films and to be covered by a surface of the cap 121. The position or space between the base 120 and the cap 121 in which a film may be disposed is referred to herein as a film holder 122 or an axial film recess. A recess 125 in the front surface of the base 120 that receives at least one film can be seen in the CT image of FIG. 1B. In some embodiments, the film is sandwiched between two flat surfaces. In in the center portion 119, the film can be recessed into a precut slot or sandwiched between flat surfaces.

The different film holders of the phantom (e.g., axial film recess 112 and peripheral film slot 122) enable film to be positioned peripherally encircling the central axis and centrally perpendicular to the central axis such that both films are simultaneously exposed to radiation.

Various radiochromic films (RCFs) that may be employed are described below in the section and subsections entitled Film Dosimetry: Available RCF models and dosimetric characteristic. The films may be exposed to a calibration pattern of radiation, and the images on the exposed films can be digitized and used for determination of calibration of the film color density with respect to dosage. Dose-Response characteristics of RCFs, useful dose ranges, and additional characteristics of RCFs are described below in the section and subsections Film Dosimetry: Dosimetric properties, Useful dose range, Energy and linear energy transfer dependence, Dose rate independence, Post-irradiation OD growth, and Advantages and limitations. The films may be exposed to treatment plan doses and the images on the films digitized and converted to measured doses using the calibration. Readout systems for reading out and digitizing image data from the films are described below in the section entitled Readout systems and data acquisition procedures. Correction to address a lateral response artifact (LRA) in some readout systems is described below in the section entitled Readout systems and data acquisition procedure. The measured doses for the treatment plan can be compared with and analyzed with respect to the treatment plan doses for QA. Additional details are presented in the section below entitled Irradiation of Calibration Films.

Film Dosimetry

The use of radiochromic film (RCF) dosimetry in radiation therapy is extensive due to its high level of achievable accuracy for a wide range of dose values and its suitability under a variety of measurement conditions. [Niroomand-Rad, A. et al. Report of AAPM Tak Group 235 Radiochromic Film Dosimetry: An update to TG-55, Medical Physics (2020) 47 (12): 5986-6025]. Accurate RCF dosimetry requires an understanding of RCF selection, handling and calibration methods, calibration curves, dose conversion methods, correction methodologies as well as selection, operation and QA programs of the readout systems.

Available RCF models and dosimetric characteristics. While conventional silver halide based x-ray films (radiographic films) are ideal for radiographic imaging using low radiation dose (few Gy), no radiographic films were commercially available to measure high doses of radiation (hundreds of Gy). In the 1980s, a new medium was developed with increased sensitivity for medical dosimetry. The basic foundation of RCF is a radiation sensitive monomer that is incorporated into a water-soluble polymer matrix coated onto a polyester base. The colorization process of RCFs is due to radiation-induced polymerization of diacetylene molecules and the formation of polydiacetylene dye polymers. As these are blue in color, the RCFs absorb light in the red and green parts of the visible spectrum. As with conventional silver halide film, the measured property of RCFs is light absorption, which is related to absorbed radiation dose. With more absorbed dose, RCFs become darker and less light is transmitted. Since the dye formation in RCF is nearly instantaneous, the color change is almost immediately visible to the naked eye. HK-810 film, for example, allows quite high spatial resolutions, up to 1200 lines per mm [Id., citing Niroomand-Rad, A. et al. Med. Phys. (1998) 25: 2093-2115]. Many newer versions of RCFs differ from the original ones in that the diacetylenes are in the form of needle-like micro-crystals about 1-211 m in diameter and 15 to 25 μm in length with an aspect ratio of about 1:10 [Id., citing Soares, C G et al. Radiochromic Film, In: Clinical Dosimetry for Radiotherapy: AAP<Summer School, Rogers, D W O and Cygler, J E Eds, Medical Physics Publishing, Madison WI (2009) Ch. 23, pp. 759-813; Williams, M. et al. Radiochromic Film Dosimetry and its Applications in Radiotherapy. In 4^{th} SSD Summer School: Concepts and trends in Medical Radiation Dosimetry (AIP Conf. Proc. 1345, Wollongong (2011) pp. 75-99; Devic, S. et al. Phys. Med. (2016) 32: 541-56]. The larger size of the crystals in newer RCFs leads to an increase in the scattering of transmitted light and reduced spatial resolution. Model EBT-XD, introduced in early 2015, also has needle-like crystals with similar 1-2 μm diameter, but with an aspect ratio closer to 1:3. [Id., citing Devic, S. et al. Phys. Med. (2016) 32: 541-56]. GafChromic HD-V2, the replacement for HD-8′0, has yellow marker dye added in the active emulsion layer to absorb light in the blue part of the spectrum, well away from the dosimetry peaks, to give a signal roughly characteristic of the emulsion thickness. [Id., citing Devic, Phys. Med. (2011) 27: 122-34; Williams, M. et al. Radiochromic Film Dosimetry and its Applications in Radiotherapy. In 4^{th} SSD Summer School: Concepts and trends in Medical Radiation Dosimetry (AIP Conf. Proc. 1345, Wollongong (2011) pp. 75-99; Devic, S. et al. Phys. Med. (2016) 32: 541-56, Bartzsch, S. et al. Medical Physics (2015) 42: 4069-79; Chen, S N et al. REev. Sci. Instrum. (2016) 87 (073301: 1-6; Devic, S. et al. Radiochromic film as a dosimetric tool for low energy proton beams. In: Proc. Of Cyclotrons (2013) Vancouver, BC Canada, WEPSH007, 1 (2013) pp. 397-99]. A thicker emulsion equates to more active component per unit area and hence greater absorbance for a constant dose.

As of 2020, the available RCFs also include those that are recommended for dosimetry in therapy, including brachytherapy, IMRT, VMAT, SBRT and SRS with photons, electrons, protons and ions of other heavier elements [Id., citing Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813; Devic, S. et al. Phys. Med. (2016) 32: 541-56] and those that are suitable for diagnostic x-ray procedures [Id., citing Devic, S. et al. Phys. Med. (2016) 32: 541-56; Tomic, N. et al. Med. Phys. (2010) 37: 1083-92; Boivin, J. et al. Med. Phys. (2011) 38: 5119-29] and interventional radiology [Id., citing Bordier, C. et al. Radiat. Prot. Dosim. (2015) 163: 306-18].

RCF models available as of 2020 include HD-V2, MD-V3, EBT2, EBT3, unlaminated EBT3, EBT3F, EBT3P, EBT3+, EBT3+P, EBT-XD, and RTQA2 designed for radiation therapy and XR-RV3, XR-QA2, XR-CT2, and XR-M2 for diagnostic radiology applications. These RCFs are manufactured in layer configurations, consisting of one or more polyester substrates, and an active layer (also called the emulsion layer) containing the active component. There are three types of configurations from bottom to top: type 1 configuration: substrate #1 then active; type 2 configuration: substrate #1, then active layer then adhesive layer then substrate #2; and type 3 configuration: substrate #1 then active layer then substrate #2.

Radiochromic films are designed with laminated structures to physically protect the active layer(s) between polyester substrates. The choices in constructing the laminates are driven by the cost and technicalities of film manufacture that are proprietary to the manufacturer. The HD-V2 and the unlaminated EBT3 film products have type 1 configuration. They are especially useful for the dosimetry of radiation that would be strongly attenuated by a polyester substrate. [Id., citing Soares, CG et al. in Clinical Dosimetry for Radiotherapy: AAPM Summer School, Rogers, S W O and Cygler, J E, Eds., Medical Physics Publishing, Madison, WI, Ch. 23, pp. 759-813; Devic, S. Phys. Med. (2011) 27: 122-34; Devic, S. et al. Phys. Med. (2016) 32: 541-56]. To protect the active layer from mechanical damage and mitigate effects from exposure to light, Type 2 and Type 3 films contain polyester substrates attached to both sides of the active layer. For example, MD-V3, EBT2, RTQA2, XR-RV3, and XR-QA2 films have type 2 configuration with either a pressure sensitive acrylic adhesive layer or a water soluble polymer to bond substrate #2 to the active layer. All members of the EBT3/EBT+product families and EBT-XD film have type 3 configuration in which both polyester substrates are directly attached to the active layer. [Id., citing Devic, S. et al. Phys. Med. (2016) 32: 541-56]. The chemistry and makeup of active layers, substrate layers and adhesive layers for various RCF are described in detail in Appendix A of Niroomand-Rad, A. et al. Report of AAPM Talc Group 235 Radiochromic Film Dosimetry: An update to TG-55, Medical Physics (2020) 47 (12): 5986-6025, which is incorporated herein by reference.

Dosimetric properties. The dosimetric properties of RCFs are described by an absorption spectrum for a given dose value and a dose-response curve for a given wavelength band. The absorption spectrum for a given dose is measured using a spectrophotometer and is a plot of optical density (OD) v. the light wavelength [Id., citing Niroomand-Rad, A. et al. Med. Phys. (1998) 25: 2093-2115; Soares, C G. Radiat. Meas. (2007) 41: S100-S116; Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813; Devic, S. Phys. Med. (2011) 27: 122-34; Williams, M. et al. Radiochromic Film Dosimetry and its Applications in Radiotherapy. In 4^{th} SSD Summer School: Concepts and trends in Medical Radiation Dosimetry (AIP Conf. Proc. 1345, Wollongong (2011) pp. 75-99; Devic, S. et al. Phys. Med. (2016) 32: 541-56, Butson, M J et al. Mat. Sci. Eng. R. (2003) 41: 61-120; Soares, C G. Radiat. Prot. Disim. (2006) 120: 100-6]. The dose-response curve is a plot of OD vs. dose for a given wavelength band of the optical densitometer readout system. Alternatively, the response curve may be a plot of the pixel values (PV) vs doses for a given output channel (red, green or blue) of a color flatbed scanner. The dose response curve is not linear regardless of the choice of OD or PV [Id., citing Niroomand-Rad, A. et al. Med. Phys. (1998) 25: 2093-2115; Soares, C G. Radiat. Meas. (2007) 41: 5100-5116; Soares, CG et al. in Clinical Dosimetry for Radiotherapy: AAPM Summer School, Rogers, S W O and Cygler, J E, Eds., Medical Physics Publishing, Madison, WI, Ch. 23, pp. 759-813, Williams, M. et al. Radiochromic Film Dosimetry and its Applications in Radiotherapy. In 4^{th} SSD Summer School: Concepts and trends in Medical Radiation Dosimetry (AIP Conf. Proc. 1345, Wollongong (2011) pp. 75-99; Devic, S. et al. Phys. Med. (2016) 32: 541-56, Butson, M J et al. Mat. Sci. Eng. R. (2003) 41: 61-120; Soares, CG. Radiat. Prot. Disim. (2006) 120: 100-6]. The OD increases with dose while the PV decreases with dose according to the relationship OD=log ₁₀ (65535/PV) for a 16 bit scan for each color channel.

The dose-response characteristics of a given RCF are dependent on many factors, including:

(1) film model, lot number, chemical composition, lamination configuration and absorption spectra; and

(2) radiation properties of particle type and energy, such as (a) photon, electron, proton; and (b) radiation energy (MeV, keV).

(1) Readout system characteristics, including: (a) wavelength range of the light source (laser, LED, white light, RGB colors); (b) wavelength range to which the light detector is sensitive; (c) film orientation on the scanner; (d) transmission vs. reflection mode; (e) temperature of the scanner glass; (f) readout system artifacts and correction; and (2) readout time after irradiation.

Useful dose range. If a choice of film models exists for the dose range of interest, one should keep in mind that for a particular RCF scanned the contrast (meaning the slope of the dose-response curve in all color channels) decreases as dose increases and approaches saturation at high dose values. [Id., citing Devic, S. Phys. Med. (2011) 27: 122-64, Grams, M P et al. Med. Phys. (2015) 42: 5782-6; Palmer, A L et al. Phys. Med. Biol. (2015) 60: 8741-52; Lewis, D F and Chan, M F. Med. Phys. (2016) 43: 643-9; Schoenfeld, A A et al. Phys. Med. Biol. (2016) 61: 5426-42; Schoenfeld, A A et al. Phys. Med. Biol. (2016) 61: 7704-24; Miura, H. et al. Appl. Clin. Med. Phys. (2016) 17: 312-22]. Therefore, if the doses of greatest interest are closer to saturation, a film with a lesser sensitivity may be a better choice. The optical resolution of the readout system should be appropriate for the dose applied to the film and be appropriate in accuracy and dose specificity. [Id., citing Butson, M J et al. Appll. Radiat. Iso. (2006) 64: 60-62]. For example, the least sensitive RCF, HD-V2, is useful for the 10-1000 Gy dose range. The MD-V3 has intermediate sensitivity, useful for the dose range of 1-100 Gy. The EBT2 and EBT3 family are useful for most clinical radiation therapy applications with dose range 0.01-20 Gy. The EBT-XC model, with a useful dose range of 0.04-40 Gy, is designed for single fraction SRS and SBRT applications.

Energy and linear energy transfer dependence. In radiation therapy, dose measurement of megavoltage (MV) radiation sources is of prime importance. However, with most sources, some fraction of the dose comes from lower energy scattered radiation. RCFs produced in November 2014 or later contained aluminum-containing compounds (alumina) within their active layer; these are designed to have minimum energy dependence and to be near water and soft tissue equivalent. Since LET is depth dependent, special attention needs to be paid to calibration and measurement of proton doses measured with RCFs by applying appropriate Let correction factors for the given depth and energy of the beam. Several methods related to LET correction of varying degrees of complexity have been reported in the literature [Id., citing Zhao, L. et al. Phys. Med. Biol. (2010) 55: N291-301; Kirby, D. et al. Phys. Med. Biol. (2010) 55: 417-33; Fiorini, F. et al. Phys. Med. Biol. (2011) 56: 6969-83; Fiorini, F. et al. Phys. Medica (2014) 30: 454-61, Gambarini, G. et al. Appl. Radiat. Isotopes (2015) 104: 192-6; Carnicer, A. et al. Radiat. Meas. (2013) 59: 225-32; Anderson, S E et al. Phys. Med. Biol. (2019) 64: 055015: 8].

Dose rate independence. The active component in current RCFs is a lithium salt of pentacosa-10, -12-diynoic acid (Li-PCDA). It polymerizes and changes color in proportion to the absorbed dose of ionizing radiation and hence it is an excellent dose integrator. Whether a given dose is applied in a single fraction, or in several smaller fractions, the net response due to the radiation is the same once sufficient time has been allowed for the expected post-irradiation OD growth to occur. Moreover, experimental data indicate that the diacetylene active components in RCFs do not exhibit dose rate dependence. [Id., citing Niroomand-Rad, A. et al. Med. Phys. (1998) 25: 2093-2115; Soares, C G. Radiat. Meas. (2007) 41: S100-S116; Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813, Williams, M. et al. Radiochromic Film Dosimetry and its Applications in Radiotherapy. In 4^{th} SSD Summer School: Concepts and trends in Medical Radiation Dosimetry (AIP Conf. Proc. 1345, Wollongong (2011) pp. 75-99; Miura, H. et al. J. Appl. Clin. Med. Phys. (2016) 17: 312-22; Borca, V C et al. Appl. Clin. Med. Phys. (2013) 14: 158-71; Rink, A. et al. Med. Phys. (2005) 32: 1140-55; Karsch, L. et al. Med. Phys. (2012) 39: 2447-55; Yao, TT et al. Radiat. Phys. Chem. (2017) 133: 37-44].

Post-irradiation OD growth. Polymerization of radiation-sensitive diacetylene monomers occurs almost immediately after the irradiation begins. The regular arrangement of the monomer molecules in a diacetylene crystal allows a linear polymerization to propagate, but owing to the slightly reduced atomic spacing in the polymer, the rate of polymerization decreases as the polymer grows. This behavior results in post-irradiation OD growth, which is characteristic of RCFs: namely, the OD increases with time, whereas the rate of OD change of an irradiated film decreases with time. The measurement protocol usually includes a waiting time to read all films of 16-24 h after irradiation, or at the same time after irradiation. To circumvent such limitation of waiting time, the user can (a) combine calibration (reference) films and measurement films in a single scan using a flatbed scanner within about half hour after irradiation of measurement films and (b) follow “one scan protocol [Id., citing Lewis, D. et al. Med. Phys. (2012) 39: 6339-50] with embedded dose correction using recalibration methods. [Id., citing Lewis, D. et al. Med. Phys. (2012) 39: 6339-50; Ruiz-Morales, C. et al. Phys. Medica (2017) 42: 67-75].

Advantages and limitations. An advantage of RCFs over the silver-based radiographic film is that they do not need post-irradiation film processing. Upon irradiation, permanent images that become darker with increasing absorbed dose are formed nearly instantaneously. Another advantage of RCF is its very low sensitivity to visible light; hence, darkrooms are not necessary and films can be cut in room light to the shape/size needed for any dosimetry purposes. However, because RCFs can be darkened if left exposed to ambient light (particularly artificially produced UV light) for an hour or longer, they should be stored in dark envelopes except when necessary for use. Further, it is recommended to store irradiated and unirradiated films at ambient room temperature (about 22° C.) or lower and to never expose them to temperatures above 60° C. In addition, the optical transmission of RCFs may vary in response to large changes in environmental relative humidity (RH), for example >60% RH. All current RCFs designed for radiotherapy contain a special marker dye. When used in conjunction with an RGB flatbed scanner and a multi-channel dosimetry method [Id., citing Micke, A. et al. Med. Phys. (2011) 38: 2523-34; Mayer, R R et al. Med. Phys. (2012) 39: 2147-55; Azori, J F P et al. Med. Phys. (2014) 41: 062101-2-062101-10; Mendez, I. et al. Med. Phys. (2014) 41: 011705-1-011704-10;], the marker dye allows the dose-response to be adjusted for small thickness differences and other nonuniformities in the active layer.

Generally, the density of radiochromic film is evaluated as the transmission of light through films, and flatbed scanners are commonly used for image acquisition of the films. Many previous studies have reported that the PVs measured using flatbed scanners show variations associated with the position on the scanner [Aikino, Y. et al. J. Radiation Res. (2021) 62 (2): 318-28, citing Paelinck, L. et al. Phys. Med. Biol. (2007) 52: 231-42; Sauer, S. et al. Med. Phys. (2008) 35: 2094-101]. Some studies reported that the causes of these lateral response artifacts are an increase in the optical path length of the film at off-center position and the interaction of the polarization of light leaving the film and the mirror system guiding the light to the charge coupled device (CCD) sensor [Id., citing van Battum, L J et al. Phys. Med. Biol. (2016) 61: 625-49; Schoenfeld, A. et al. Phys. Med. Biol. (2016) 61: 7704-24]. This effect leads to incorrect measured data, and the impact is not negligible especially for patient-specific QA of large targets.

The Lateral Response Artifact (LRA) can be substantial in causing uncertainty in dose determination [Niroomand-Rad, A. et al. Report of AAPM Tak Group 235 Radiochromic Film Dosimetry: An update to TG-55, Medical Physics (2020) 47 (12): 5986-6025, citing Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813, van Battum, U et al. Phys. Med. Biol. (2016) 61: 625-49; van Battum, U et al. Med. Phys. (2008) 35: 704-16, Lewis, D. and Chan M F. Med. Phys. (2015) 42: 416-29]. This artifact imposes restrictions on the size and scanning orientation of films in flatbed scanners. In addition, off-center film placement of the scanner, as well as doses >5Gy, and single-channel dosimetry can introduce dose uncertainties >10% [Id., citing Lewis, D. and Chan M F. Med. Phys. (2015) 42: 416-29]. This effect can be mitigated through the use of multi-channel dosimetry and can be eliminated through a one-time procedure to characterize the effect for a given film model using a particular scanner. The resulting correction coefficients can be applied to remove the artifact from any subsequent image obtained for that film model on the scanner. [Id., citing Lewis, D. and Chan M F. Med. Phys. (2015) 42: 416-29].

Turning again to phantom 100, FIGS. 1B and 1C include measured distances associated with the phantom employed for the Example, which is described below. One of ordinary skill in the art in view of the present disclosure will recognize that phantoms disclosed herein are not limited to the dimensions in FIGS. 1B and 1C, are not limited to the relative dimensions depicted in FIGS. 1A-1C, and are not limited to the shapes depicted in FIGS. 1A-1C. For example, in some embodiments, an outer peripheral ring portion may be connected with a center portion by some structure or structures other than a support disk. In some embodiments, the center portion may have a larger diameter and/or the inner ring may have a smaller inner diameter reducing the open space between the peripheral ring portion and the center portion. In some embodiments, there may be no open space between the peripheral ring portion and the center portion. In some embodiments, at least a portion of the inner ring and at least a portion of the center portion may be combined, affixed to each other, or integral with each other.

In use, radiochromic film is positioned in the peripheral slot 112 and in the axial recess 122 of the phantom. In some embodiments, the peripheral film in the peripheral slot 112 may completely encircle the central axis 109. In some embodiments, the peripheral film may only partially encircle the central axis 109. In some embodiments, the peripheral film may only extend over 180 degrees of the inner ring.

The films in the phantom are exposed to radiation according to a radiation plan. The films are then removed from the phantom and images on the film are converted to digital images (e.g., using a scanner). When performing calibration for the film, the radiation plan may be a calibration radiation plan or a calibration dose plan, and one or more color channels (e.g., red, green, blue) of the image are used to relate density or intensity of the color in the image to a dose received by the film for calibration. In some embodiments, the calibration dose plan may be determined by the system, at least in part, based on a patient-specific radiation treatment dose plan. For example, in some embodiments, ranges of doses in some portions of the calibration dose plan may correspond to ranges at corresponding locations in the patient-specific radiation treatment dose plan. In some embodiments, an isocenter of the calibration dose plan may correspond to an isocenter of the patient-specific dose plan. In some embodiments, the peripheral film or films are used for calibration. In some embodiments, patient-specific calibration plans may be created to cover the patient range of doses with the same beam energy. In some embodiments, the peripheral film or films and the axial film are used for calibration. In some embodiments, the axial film is used for calibration. When using the phantom for QA, the films in the phantom may be exposed to radiation according to a radiation treatment plan. The films are then removed and converted to digital images (e.g., using a scanner). Film calibration is used to transform the density or intensity of the color in one or more color channels of the image into dose received at that portion of the film. The dose received at various portions or locations of the central axial film, of the one or more peripheral films, or both may be compared with at least one planned treatment dose profile for QA purposes, which can include generating a gamma index for a specified portion of the phantom.

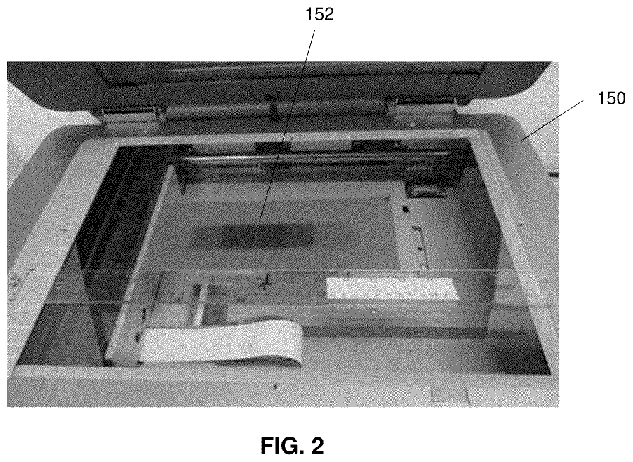

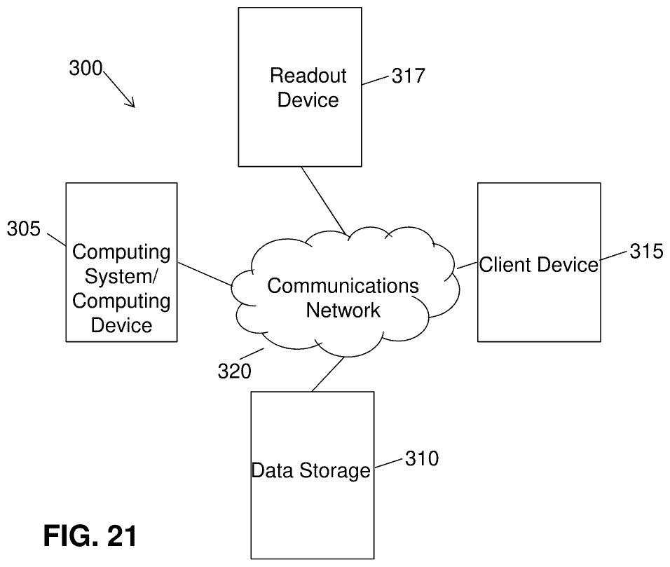

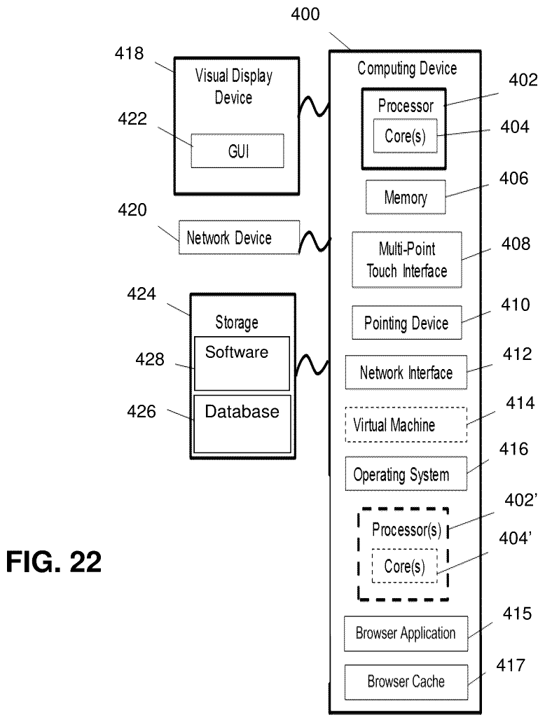

In accordance with embodiments of the present disclosure, an exemplary system for SBRT is provided. The system includes a phantom as described herein, which serves as a medium having a specific material and geometry with which the radiation beam or beams interact(s) and generate(s) either a directly detected signal or is modified and subsequently measured, producing an indirectly detected signal. In some embodiments, the system includes at least one detector (e.g., at least one radiochromic film) with which the radiation interacts and that is used to record the interaction. After exposure to radiation, the film(s) is/are removed from the respective film holder of the phantom and an image on each film is digitized (e.g., using a color scanner). In some embodiments, the system includes the device used to digitize the image (e.g., the scanner). For example, see FIG. 2 with an image of a color scanner for scanning a peripheral film exposed to a calibration dose plan. In other embodiments, the system does not include the device used to digitize the image. In some embodiments, the system includes at least one processing device or system including at least one processor for processing and/or analyzing the digitized image data. The resulting digitized image data is processed to yield color intensity in a certain channel (e.g., red, blue or green) and/or a combination of channels at each point in 2D, and/or to yield color intensity versus distance along a line. The at least one processing device or system can receive as input the digitized color image data and/or the yielded color intensity, and can analyzes the input film image data. In some embodiments, the at least one processing device or system can also receive or generate information regarding a calibration dosage plan or a treatment dosage plan. In some embodiments, the at least processing device can be a computing device or a computing system (e.g., a smart device, a computer, or the like), programmed to conduct a method for analyzing the input digitized film image data. Further information regarding systems, networks, computing systems and computing devices that may be employed in some embodiments or in which some embodiments may be implemented is described below with respect to FIGS. 21 and 22.

In some embodiments, the method can be implemented, at least in part, in a programming and numeric computing platform (e.g., MATLAB® from MathWorks, Inc.). In some embodiments, an imaging toolbox in a programming and numeric computing platform may be employed for analyzing the digital image data from the film. A method or methods programmed into the at least one processing device or system can be used for accurate dose calibration with verification, scanner correction, and a versatile analysis tool (such as Gamma Index in specific geometrical regions of interest) in accordance with some embodiments. The at least one processor/processing device can therefore amplify, digitize and scale/convert signals from the detector (e.g., the film) to the appropriate dose quantity, in some instances through application of calibration coefficients. In some embodiments, the system can include a data handler/software to manage the measured signal and position data.

In some embodiments, scanner and phantom uniformity may be evaluated. The scanner and phantom uniformity can be evaluated by comparing a uniform field plan dose with the corresponding film density. The dose to density calibration curve can be loaded or input into the system, and the uniformity plan dose in the central plane can be read. In some embodiments, a separate flat water equivalent phantom commonly used in radiotherapy may be employed to evaluate scanner uniformity. In some embodiments, this uniformity plan dose is measured with the axial film. The plan dose can be converted to density, and the red channel of the uniformity film can be read. The ratio of film density to plan density in the corresponding profiles can be generated, and a best linear fit to profile can be determined with the slope recorded and stored in the system. The linear fit can be used for longitudinal laterality correction, if needed.

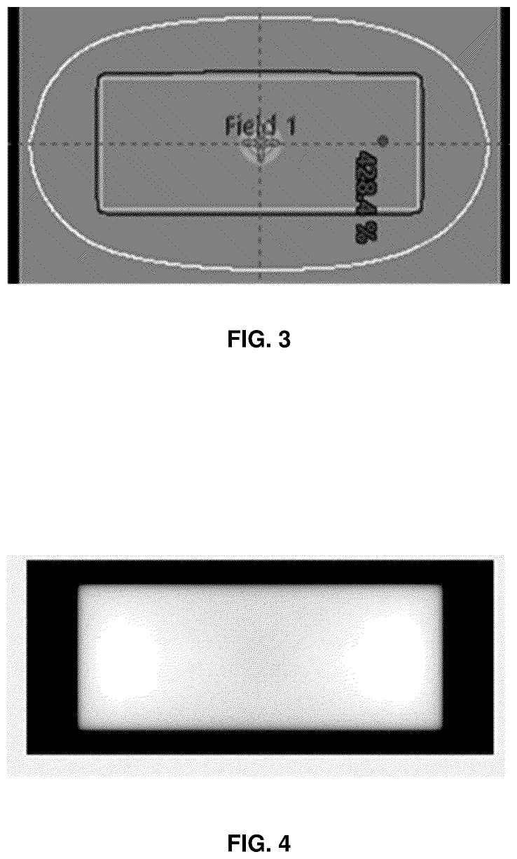

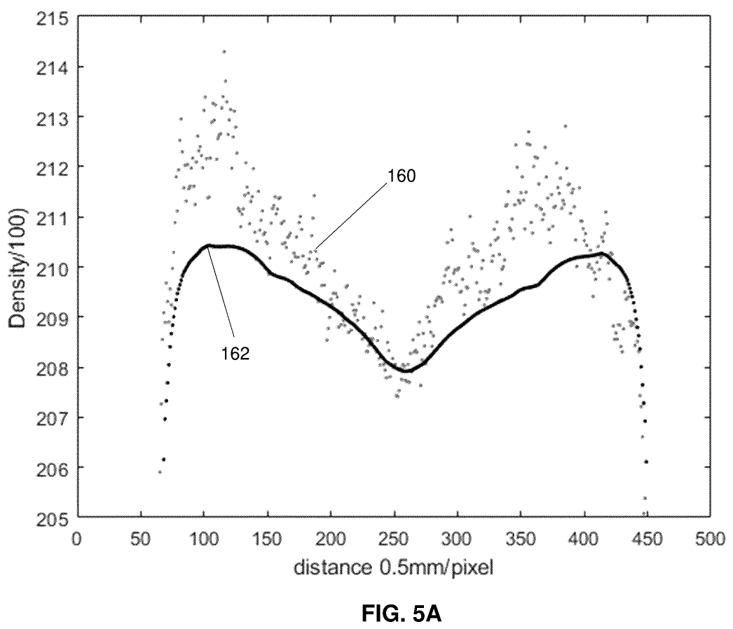

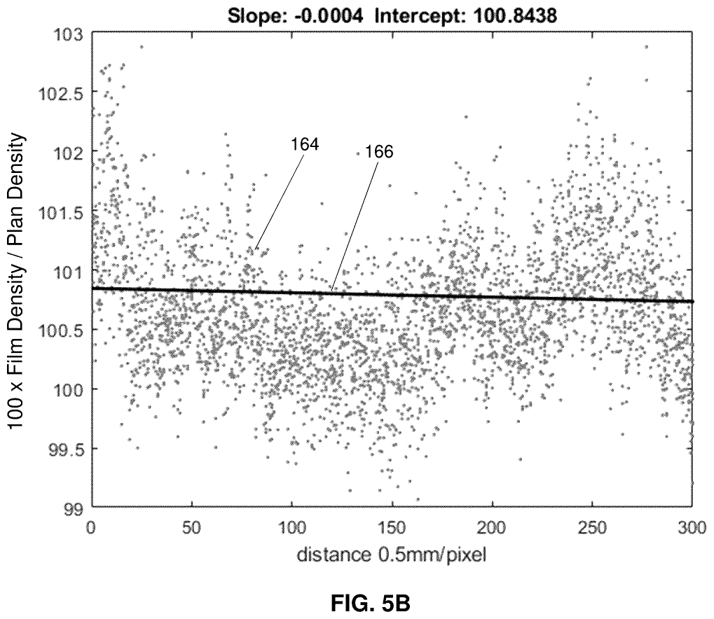

For example, a uniformity correction plan for determining uniformity of the scanner may include a rectangular area of constant dose incident at the central plane (see, e.g., FIG. 3). An exemplary image of the peripheral film after exposure to the uniformity correction plan in a separate flat solid water phantom commonly used in radiotherapy at a depth of 1.5 cm with 10 cm of backscatter and a 6MV beam is shown in FIG. 4. A density as a function of distance for the digitized data from the exposed film (e.g., dots in FIG. 5A) is compared to that of the uniformity correction plan dose (e.g., black line in FIG. 5A) to determine if any corrections are needed for uniformity. FIG. 5B shows a graph of the Film Density/Density percent error as a function of distance along the peripheral film.

In some embodiments, a calibration curve may be generated to convert the signal intensity from the digitized film image in at least one of the color channels (e.g., the red channel) to the corresponding dose at the peripheral film (e.g., from the film disposed in the peripheral film holder, slot or space 112 between the outer and inner rings 104, 110). Although the film can provide red, green, and blue channels (and in some embodiments each of the channels can be used), in some embodiments, the system can rely only on the red channel to generate the calibration curve.

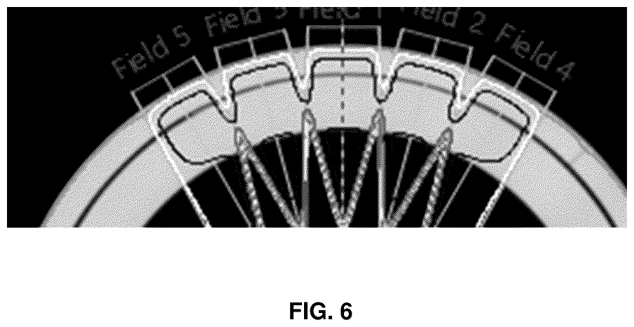

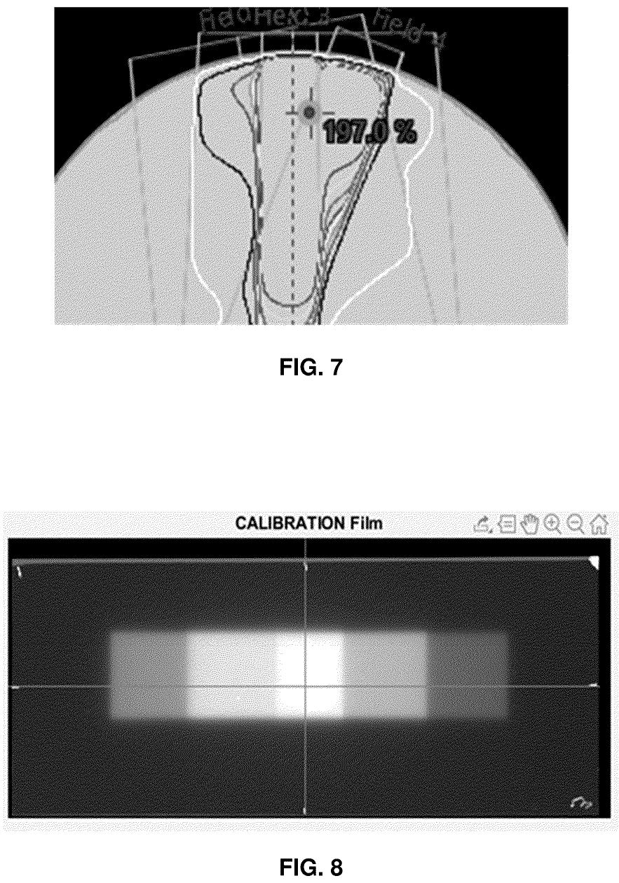

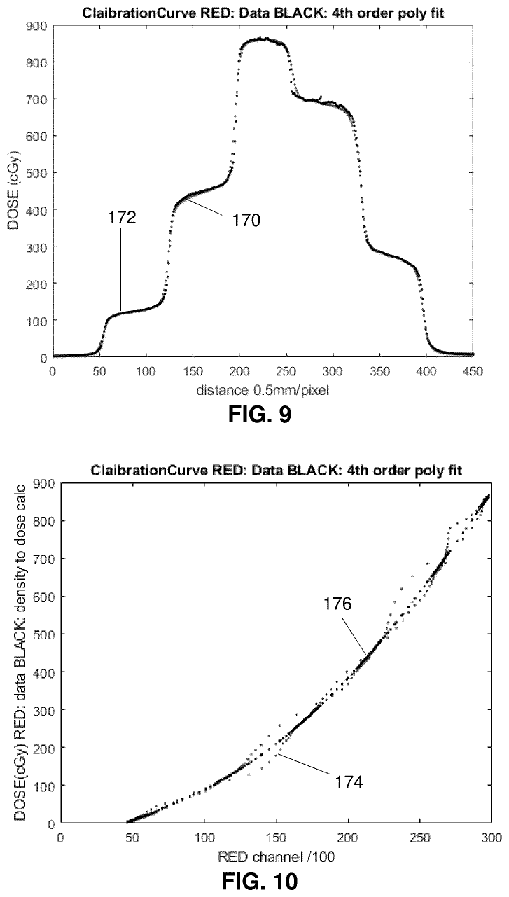

In some conventional systems, only a few select dose points are used to determine a density to relationship over a wide range of doses. In contrast, in some embodiments of the present disclosure, a static calibration plan is generated in the phantom with the actual clinical setup including isocenter and beam energy. FIG. 6 depicts an exemplary plan generated with multiple static fields for calibration of the phantom at the peripheral film. A statistically large number of sample points may be employed to ensure accuracy and robustness for the calibration curve. Converting measured film density to delivered dose is accomplished by creating a density to dose calibration curve, as shown in FIG. 7 for the calibration film in FIG. 8. The calibration film in FIG. 8 was generated by exposure of the peripheral ring film to the calibration plan radiation. FIG. 9 shows the plot of dose versus distance measured in the film (170) (red) and predicted (172) (black, 4^{th} order poly fit) for the calibration plan. FIG. 10 shows a dose v. distance plot for the measured film (red) (174) and for predicted (176) for the calibration plan to determine the calibration curve. In some embodiments, the polynomial coefficients for the calibration curve are electronically stored in a database associated with the system.

In some embodiments, analysis of a patient-specific plan may be conducted. This may include determining a gamma index for a specific geometrical region of interest, which may be referred to herein as determining a specific gamma index, that may be based on measurements from at least one peripheral film encircling the central axis, measurements from at least one central axis film, or from both. Information regarding the treatment plan can be imported from the treatment planning system, and planned doses at the central axial file and/or at the one or more peripheral films can be calculated. After the phantom is exposed to radiation corresponding to the treatment plan, the film can be read and converted to dose, and the gamma index can be computed from the planned dose distribution and the measured dose distribution. Information regarding calculation of the gamma index is presented below in the section entitled Gamma Index. The methods for determining a gamma index described herein are versatile enabling determination of a generic or general gamma index as well determination of one or more specific gamma indices each corresponding to a geometrical region of interest (e.g., a clinically relevant area). The methods employing film enable fine resolution for both the reference and the measured data. The methods employing both one or more peripheral films and a central axis film enable fine resolution for a peripheral region as well as for a central region and enable determination of a gamma index including data from a peripheral region. Further, the methods enable a references dose to be set to different values in different specified regions. For example, the gamma index can be computed in a specific region such as the quadrant with x<0 and y>0. Also the denominator term corresponding to the global Gamma Index reference dose, commonly the maximum plan dose, can be specified to any other value such as a critical structure tolerance dose. Further, the determination of the gamma index may avoid the use of complex interpolation calculations due to the fact that both the reference plan and the measured film have fine spatial resolution.

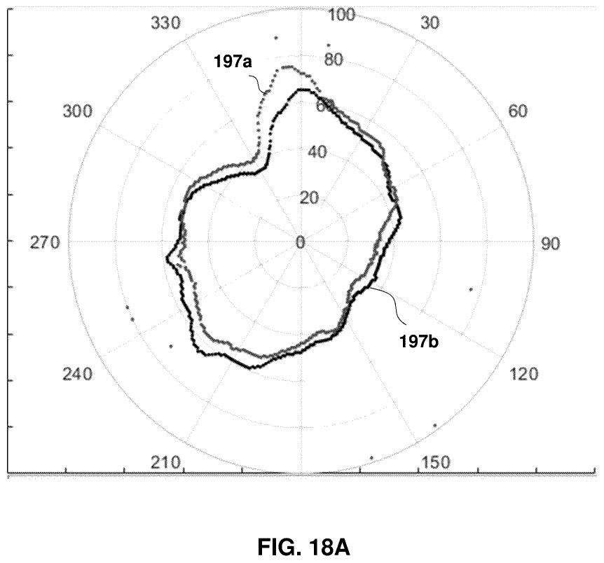

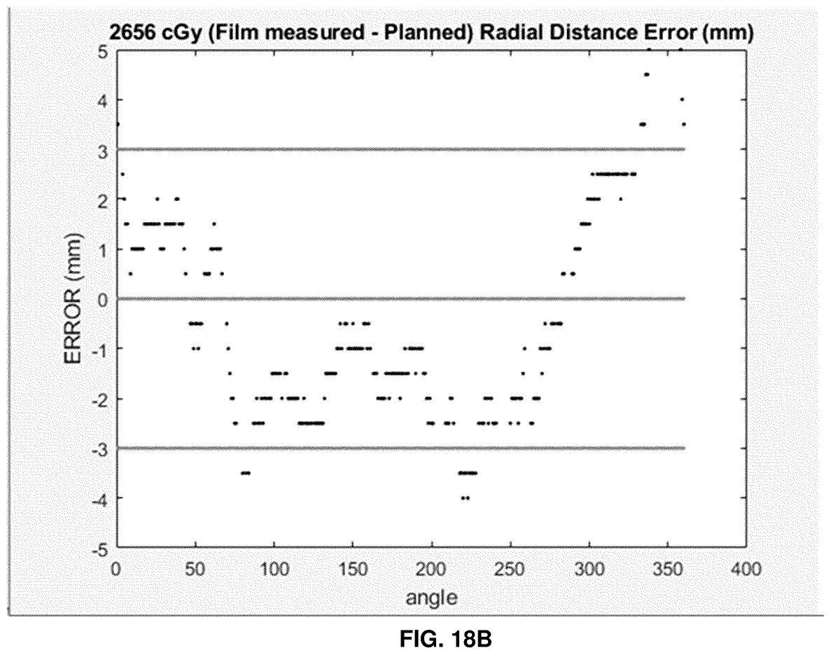

Some embodiments provide methods for generating polar coordinate plots of specific isodose lines for plan dose and measured film dose in a clinically relevant axial plane for comparison (see FIGS. 18A, 19A, 20A and accompanying explanation with respect to the Example). The difference in radial distance between the plan dose and corresponding measured film dose for each angle in one-degree increments is similar to DTA in gamma calculations. However, the visual plot can demonstrate where the deviations are located relative important or critical anatomical structures. The radial differences for various angles are depicts in FIGS. 18B, 19B, and 20B and described below with respect to the Example). The maximum of the absolute value of all the radial differences is analogous to the radial Hausdorff Distance (rHD).

In some embodiments, radial Hausdorff Distance (rHD) may be determined based on the planned dose distribution and the measured dose distribution at the central axis plane/film for various angles with respect to the central axis. A method for determining the rHD includes selecting an isodose level, and searching in the plan dose axial plane for the radial distance at which the selected isodose occurs for a particular angular orientation. The angular orientation is incremented and the search in the plan dose axial plane for the radial distance at with the selected isodose occurs is repeated for the incremented angular orientation. This is repeated until all 360 degrees of orientation have been covered. This process is repeated for the film dose axial plane. The rHD as a function of orientation is the difference between plan dose radial distance and the film dose radial distance at that orientation divided by two. The single value rHD is the maximum value of rHD over all of the orientations.

Readout Systems and Data Acquisition Procedures

A radiochromic dosimetry system consists of two integral parts, namely, RCF and a readout system (such as a spectrophotometer, densitometer or scanner). The optimal quantitative performance of RCF dosimetry system can be achieved by selecting the film model with appropriate sensitivity to the intended type and energy of radiation and by matching the readout system with its ability to distinguish the radiolytic color change.

The dosimetric methods used to quantify response of conventional silver-halide film can be applied to RCFs as well. These include spot densitometers and two-dimensional optical densitometers. An important consideration for all RCF readout systems is to optimize (a) the wavelength of light source and (b) light detector sensitivity to the absorption peaks of the RCF that is being read. The output of densitometer and spectrophotometer readout systems is a value in OD units, which is the inverse logarithm of the light transmission. However, in flatbed scanner systems the primary readout is PV in units proportional to light transmission, but there are some options to convert the PV to OD. Ideally, the delivered absorbed dose should be a linear function of the measured OD, but it should be pointed out that there is a non-linear relationship between absorbed dose and transmitted light and the change in OD is not linear with dose. Niroomand-Rad, A. et al. Report of AAPM Tak Group 235 Radiochromic Film Dosimetry: An update to TG-55, Medical Physics (2020) 47 (12): 5986-6025, citing Soares, C G. Radiat. Meas. (2007) 41: S100-S116; Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813, Williams, M. et al. Radiochromic Film Dosimetry and its Applications in Radiotherapy. In 4^{th} SSD Summer School: Concepts and trends in Medical Radiation Dosimetry (AIP Conf. Proc. 1345, Wollongong (2011) pp. 75-99, Devic, S. et al. Phys. Med. (2016) 32: 541-56; Soares, C G. Radiat. Prot. Dosim. (2016) 120: 100-6.]

2D film scanner types. There are several commercially available choices of two-dimensional optical scanners. The 2D scanners measure light absorption by the active layer of the RCF, either in transmission or reflection mode, with the output being a digital image. [Id., citing Niroomand-Rad, A. et al. Med. Phys. (1998) 25: 2093-2115; Soares, C G. Radiat. Meas. (2007) 41: S100-S116; Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813, Williams, M. et al. Radiochromic Film Dosimetry and its Applications in Radiotherapy. In 4 th SSD Summer School: Concepts and trends in Medical Radiation Dosimetry (AIP Conf. Proc. 1345, Wollongong (2011) pp. 75-99; Devic, S. et al. Phys. Med. (2016) 32: 541-56]. The development of multi-channel dosimetry [Id., citing Micke, A. et al. Med. Phys. (2011) 38: 2523-34; Mayer, R R et al Med. Phys. (2012) 39: 2147-55; Azorin, J F P, et al. Med. Phys. (2014) 41: 062101-1-062101-10] with its inherent advantage of compensating for non-uniform thickness of the active layer has made color flatbed scanners particularly attractive.

Translational type: scanning spot densitometer and drum scanner. Scanning spot densitometers are based on translating a small light source and detector assembly relative to the film object in directions (x and y) that are controlled by a computer. Film is positioned and fixed on or slightly above a glass bed. The light source can be He-Ne laser or red LED. The overall scan time depends on the dimension of the area of interest, step size, stepping motor control, and computer interface protocol. The spatial resolution ranges from 0.05 to 1 mm. [Id., citing Niroomand-Rad, A. et al. Med. Phys. (1998) 25: 2093-2115; Dempsey, J F et al. Med. Phys. (1999) 26: 1721-31]. Although the scanning speed is low, the scanning spot densitometer is usable for small areas of interest needed in IVBT dosimetry.

Drum scanner. Van Battum, L J et al. Phys. Med. Biol. (2016) 61: 625-49 reported the use of a drum scanner with EBT3 film. They showed that the output from the drum scanner does not exhibit any LRA due to the absence of minors in the optical system, and also from the lack of optical path length variation since the light path is always perpendicular to the film. However, the high cost of a drum scanner makes it impractical for routine RCF scanning.

Imaging type. An imaging type densitometer consists of a stationary high-resolution CCD camera 2D CCD array) mounted on a vertical sliding arm above a separate stationary LED light box. The CCD camera and light box are in a light tight cabinet. The RCF is positioned on the top surface of the light box, and then covered by a glass plate. The camera captures transmitted light through the film. A 2D image is acquired, with the output from a 2D array of OD values of 242×375 or 512×512 pixels, depending on the camera type. [Id., citing Devic, S. et al. Med. Phys. (2004) 31: 2392-2401; Chiu-Tsao, S T et al. Med Phys. (2008) 35: 3787-99] The field of view (FOV) is adjustable based on the height of the camera and the appropriate lens and aperture in the camera. Due to the fixed number of pixels in the image acquired, the spatial resolution varies with the FOV. Since the films (single sheet or multiple small pieces) are stationary on the light box during a scan, all films (experimental, calibration, and background) can be simultaneously imaged as long as they are within the FOV. [Id., citing Chiu-Tsao, ST et al. Med Phys. (2008) 35: 3787-99]. Before scanning starts, the camera and light box top surface is cooled down to and stabilized at about 0° and 20° C., respectively, for consistent data acquisition. Before scanning a series of films, a “flatfield” image is acquired without an object on the light box. The flatfield image is used to correct for light source non-uniformity, lens optical properties, and pixel-to-pixel variations in CCD sensitivity. The subsequent image acquired with a film present is then corrected with the flatfield image to yield an OD map in 2D. [Id., citing Chiu-Tsao, ST et al. Med Phys. (2008) 35: 3787-99]. The output OD data are 16 bit in depth. The LED light sources are available in several different wavelengths. [Id., citing Chiu-Tsao, S T et al. Med Phys. (2004) 31: 2501-8; Chiu-Tsao, ST et al. Med Phys. (2008) 35: 3787-99; Chiu-Tsao, S T et al. Med. Phys. (2005) 32: 3350-54; Chiu-Tsao, S T, et al. Appl. Radiat. Isot. (2014) 92: 102-114]. The 665±10 nm red, 520±20 nm green, and 465±14 nm blue light colors are optimal for matching the absorption peaks of the film models HD-810, MD-55, HS, and XR.17, [Id., citing Devic, S. et al. Med Phys. (2007) 34: 112-118] The OD is highest for red, intermediate for green and lowest for blue colors in these earlier film models. For different colors, the individual light boxes must be repositioned at the bottom of the cabinet and parallel port and power plugs properly connected. When the camera is moved or the lens or the light box is changed, the lens must be refocused, which is an involved procedure. [Id., citing Soares, C G. Radiat. Meas. (2007) 41: S100-S116]. The data for only one color channel at a time can be acquired, in contrast with the simultaneous data acquisition of all three color channels using flatbed scanners

Combined translational and imaging type. The combined translational and imaging type 2D readout systems have much faster scan speed than those of translational type. The film object is imaged in one direction while the light source and detector array are translated in the perpendicular (scanning) direction. [Id., citing Niroomand-Rad, A. et al. Med. Phys. (1998) 25: 2093-2115; Soares, C G. Radiat. Meas. (2007) 41: S100-S116; Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813]. Most film scanners of the combined translational and imaging type employ a linear CCD array. [Id., citing Niroomand-Rad, A. et al. Med. Phys. (1998) 25: 2093-2115; Soares, C G. Radiat. Meas. (2007) 41: S100-S116].

Color flatbed scanners: reflection and transmission scanning. The light source in most color flatbed scanners is a broad-spectrum fluorescent tube or a white LED array, with light emission across all visible wavelengths. The optical components of flatbed scanners consist of a light source, minors, lenses, and a linear CCD sensor array. During a scan, the RCF stays stationary on the scanner glass bed while the light source and sensor assembly move in a direction perpendicular to the light source and sensor array axis. [Id., citing Soares, C G. Radiat. Meas. (2007) 41: S100-S116; Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813]. In contrast with VIDAR scanners, color flatbed scanners all have RGB capability, mostly 16 bit per color channel. The CCD array has three parallel lines of sensors, each one with a red, green, or blue optical filter, which are separately sensitive in the red (600-700 nm), green (500-600 nm), and blue (400-500 nm) wavebands. Such RGB capability is suitable for the application of the multi-channel dosimetry method [Id., citing Micke, A. et al. Med. Phys. (2011)38: 2523-34; Mayer, R R et al. Med. Phys. (2012) 39: 2147-55; Azorin, J F P et al. Med Phys. (2014) 41: 062101-1-062101-10].

Reflection scanning. All flatbed scanners are capable of scanning in reflection mode where a light source in the base of the scanner is used to illuminate the front of the film. [Id., citing Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813; Devic, S. et al. Phys. Med. (2016) 32: 541-56; Papaconstadopoulos, P. et al. Medical Physics (2014) 41: 122101-1 — 122101-6]. The vendor provides a reflective white plastic sheet to be placed behind (above) the film. When performing reflection scanning of RCF models with a white substrate (e.g. RTQA2, XR-RV3, and XR-QA2) the orange side of the film faces the glass window in the scanner base. Transparent films (all EBT, MD and HD types) can also be scanned in reflection mode. Light reaching the detector has either been reflected from the white film substrate, or from the white insert and has passed twice through the active layer of the film. While this enhances film response, a portion of the signal comes from light reflected from the front surface of the film. The active layer does not attenuate this signal. For film irradiated to relatively low doses, the front surface reflection is only a minor portion of the total signal. With increased dose, the active layer absorbs more light while the front surface reflection is constant. Ultimately, the front surface reflection becomes a substantial portion of the signal. This reduces the slope of the dose—response curve and limits the dynamic range of EBT2/3 films to about 2.5 Gy (red channel) and 8 Gy (green channel) for reflection mode. [Id., citing Papaconstadopoulos, P. et al. Medical Physics (2014) 41: 122101-1-122101-6]. While the dynamic dose range is narrower for reflection mode compared with transmission mode, the film sensitivity is enhanced at lower doses.

Transmission scanning. All flatbed scanners are capable of scanning in reflection mode where a light source in the base of the scanner is used to illuminate the front of the film [Id., citing Soares, C G et al. in Clinical Dosimetry for Radiotherapy: AAPM Summer School, Rogers, S W O and Cygler, J E, Eds., Medical Physics Publishing, Madison, WI, Ch. 23, pp. 759-813; Devic, S. et al. Phys. Med. (2016) 32: 541-56; Papaconstadopoulos, P. et al. Medical Physics (2014) 41: 122101-1-122101-6]. The vendor provides a reflective white plastic sheet to be placed behind (above) the film. When performing reflection scanning of RCF models with a white substrate (e.g. RTQA2, XR-RV3, and XR-QA2) the orange side of the film faces the glass window in the scanner base. Transparent films (all EBT, MD and HD types) can also be scanned in reflection mode. Light reaching the detector has either been reflected from the white film substrate, or from the white insert and has passed twice through the active layer of the film. While this enhances film response, a portion of the signal comes from light reflected from the front surface of the film. The active layer does not attenuate this signal. For film irradiated to relatively low doses, the front surface reflection is only a minor portion of the total signal. With increased dose, the active layer absorbs more light while the front surface reflection is constant. Ultimately, the front surface reflection becomes a substantial portion of the signal. This reduces the slope of the dose—response curve and limits the dynamic range of EBT2/3 films to about 2.5 Gy (red channel) and 8 Gy (green channel) for reflection mode. [Id., citing Papaconstadopoulos, P. et al. Medical Physics (2014) 41: 122101-1-122101-6]. While the dynamic dose range is narrower for reflection mode compared with transmission mode, the film sensitivity is enhanced at lower doses.

According to some embodiments, an option for RCF dosimetry using flatbed scanners is the ‘one-scan’ protocol with embedded recalibration methods. [Id., citing Lewis, D. et al. Med Phys. (2012) 39:6339-6350; and Ruiz-Morales, C. et al. Phys Medica. (2017) 42:67-75]. In the “one-scan” protocol, the user irradiates the reference film to a known dose similar in magnitude to the maximum expected on the measurement film. [Id.] The use of a recalibration method compensates for inter-scan variability, changes in film response due to varying environmental conditions, and post-irradiation OD growth of RCF. It saves time between irradiation and evaluation and eliminates uncertainty caused by scanner and temperature variability between separate calibration and measurement scans. [Id., citing Soares, C. G. et al., Clinical Dosimetry for Radiotherapy: AAPM Summer School, Medical Physical Publishing, Madison, WI, ISBN 9781888340846 (2009), Cap. 23, pp. 759-813].

The longitudinal response of flatbed scanners, that is, response parallel to the scan direction, is very stable with no substantial impact on dose uncertainty. [Id., citing Fiandra, C. et al. Med. Phys. (2006) 33:4314-4319; Saur S. et al. Med. Phys. (2008) 35:3094-3101; Lewis, D. et al. Med. Phys. (2015) 42:5692-5701; Poppinga, D. et al. Med. Phys. (2014) 41:021707.021707-1-021707-8]. However, when a large film (with dimension close to the scan window size) is scanned, the LRA can be a substantial source of dose uncertainty. [Id., citing Khachonkham, S. et al. Phys. Med. Biol. (2018) 63:065007. 1-11; Schoenfeld, A A et al. Phys. Med. Biol. (2014) 59:3575-3597]. It has been reported the red color channel response of EBT model on flatbed scanners was dependent on lateral position, that is, the position of the film perpendicular to the scan direction. [Id., citing Fiandra, C. et al. Med. Phys. (2006) 33:4314-4319; Lynch, B D et al. Med. Phys. (2006) 33:4551-4556; Paelinck, L. et al. Phys. Med. Biol. (2007) 52:231-242; Menegotti, L. et al. Med Phys. (2008) 35:3078-3085]. Films positioned further from the center of the scanner exhibit an overestimate of dose—response, particularly for doses >2 Gy. Some have reported that the magnitude of LRA of EBT3 films is greater for higher dose (LRA Correction). [Id. citing Lewis, D. et al. Med. Phys. (2015) 42:416-429]. The LRA is less pronounced in the green and blue color channels, as compared with the red channel, probably due to the weaker optical absorption in those spectral bands. They also found that while the specific characteristics of the LRA were scanner-dependent, they were independent of the EBT3 film production lot. Lewis et al. later reported that the LRA of EBT3 and EBT-XD films having the same PV (but different doses) for a given color channel are indistinguishable. [Id.] Grams et al. compared the LRA of EBT3 and EBT-XD for the same doses (10, 20, and 30 Gy) and found that EBT-XD had less LRA effect than EBT3. [Id., citing Grams, M P et al. Med. Phys. (2015) 42:5782-5786].

van Battum et al. attributed causes of the LRA to differences in optical path length inside the film with increasing distance from the center of the scanner, and how the scanner's optical minor system responds to light polarization from the film. [Id., citing van Battum, L J et al. Phys. Med. Biol. (2016) 61:625-649]. The LRA stems from differences in reflectivity from the multiple mirrors in the optical train of flatbed and VIDAR scanners where the angle of incidence changes with distance from the center of the scanner. Since the polarization of the transmitted light increases at higher film doses, the LRA is also dose-dependent. The higher PV (lower OD) in the green and blue channels results in reduced LRA in those spectral bands. Schoenfeld et al. studied the optical characteristics of EBT3 and EBT-XD film, finding a dependence of LRA on path length, partial polarization, and light scattering, and stated that all three effects are of equal magnitude. For small (<1 cm) fields with steep dose gradients, a “cross-talk” effect was found to cause dosimetric uncertainty. [Id. citing Schoenfeld, A A et al. Phys. Med. Biol. (2016) 61:5426-5442; Schoenfeld, A A et al. Phys. Med. Biol. (2016) 61:7704-7724; van Battum, L J et al. Phys. Med. Biol. (2016) 61:625-649]. This effect results from light originating from adjacent light source element(s), due to the linear light source in a flatbed scanner.

Spectrophotometer. For RCF applications, a spectrophotometer is used to measure the absorption spectrum as a function of dose. [Id., citing Devic, S. et al. Med. Phys. (2007) 34:112-118; Butson, M J et al. Phys. Med. Biol. (2005) 32:3350-3354; Alnawaf, H. et a. Radiat. Meas. (2010) 45:129-132]. Since the measured OD in densitometers is a function of the wavelengths at which the absorbance is sampled, the knowledge of the absorption spectrum of the RCF sensitive layer is useful in designing or selecting the most optimal readout system. Although the spectrophotometer is not commonly used in the clinic, it is important for clinical physicists to be aware of the absorption spectrum of RCF, as obtained with a spectrophotometer, in order to make the best choice of color channel(s) or light sources in readout systems.

Irradiation of Calibration Films

RCF dosimetry requires four major steps; (a) accurate data acquisition and film readout (Section 2), (b) proper film calibration (this section), (c) proper documentation and standardized procedures, and (d) proper data processing and analysis in establishing post-irradiation dose—response OD. [Id., citing Devic, S. et al. Phys. Med. (2016) 32:541-556].

In principle, any radiation field for which the absorbed dose rate is known can be used for the irradiation of calibration films. However, there are practical considerations which may limit the utility of a given radiation type. For example, the dose rate should be sufficient to irradiate all films in a reasonably achievable time frame. For proper film calibration, it is important that the radiation field should deliver a uniform dose (with variation in dose profile not exceeding 3%) over a region of interest (ROI) in the calibration films. The ROI should be large enough that a histogram of all PVs results in a normal distribution, which can be adequately described in terms of standard deviation. The dose rate should be sufficient to irradiate all films in a reasonably achievable time frame. Calibrated linear accelerator (linac) and 60 Co units are suitable for accurate film calibration. Depending on the particular application and film type, the energy response of the film should be considered by using a radiation beam that has the same energy characteristics as the one used in experimental conditions. [Id., citing Massillon-JL, G. et al. Int. J. Med. Phys. Clin. Eng. Rad. Oncol. (2012) 1:60-65].

Data Processing and Analysis.

The first step in processing and analyzing the data from RCF is to convert the information contained within the film to a digital format in terms of OD or PV in some embodiments. For flatbed scanners the image data obtained from film readout procedures is characterized utilizing 48-bit technology. Image data of 16-bit depth is obtained from each color channel of the scanned image. The resulting data files can be in various formats depending on the system used. The most commonly used method of scanning RCFs is to use a flatbed scanner, and therefore the typical data format is the uncompressed TIFF. [Id., citing Soares, C G et al. Radiat. Meas. (2007) 41:S100-S116; Soares, C G et al. Clinical Dosimetry for Radiotherapy: AAPM Summer School, Medical Physica Publishing, Madison, WI, ISBN 9781888340846 (2009) Chap. 23, pp. 759-813; Williams, M. et al. 4 th SSD Summer School: Concepts and Trends in Medical Radiation Dosimetry, AIP Conf. Proc. 1345, Wollongong (2011) pp. 75-99]. Each TIFF file contains a header block followed by binary image data, which can be viewed using a TIFF tag viewer. Such viewers are helpful since the ability to determine where important information is located within the header is useful for users who wish to write code capable of obtaining color channel information. Programming software products such as MATLAB (MathWorks, Natick, MA), 48, 100 or IDL (Exelis VIS, Boulder, CO) 103, 105 are suitable to create film analysis software. Alternatively, software such as ImageJ, 151 ImageMagick (imagemagick.org) and radiochromic.com are available and have also been used for image analysis. Proprietary software designed for RCF analysis such as FilmQAPro (Ashland), RIT113 (RIT), OmniPro I'mRT (IBA Dosimetry), Mephysto mc2 (PTW), Film Analysis (Sun Nuclear), TomoTherapy Film Analyzer152 (Accuray Inc., Madison, WI), and DoseLab Pro (Mobius) have also been used.

As with any image analysis, RCF dosimetry data are subject to noise. [Id., citing Vera Sanchez, J A et al. Phys. Medica. (2016) 32:1167-1174]. The origin of the noise can be from many factors, such as film non-uniformity, dust, or fingerprints on the film itself, [Id., citing Butson, M J et al. Mat. Sci. Eng. R. (2003) 41:61-120; Palmer, A L et al. J. Appl. Clin. Med. Phys. (2014) 15:280-296]. Newton's rings, or scanner fluctuations. [Id., citing Williams, M. et al. 4th SSD Summer School: Concepts and Trends in Medical Radiation Dosimetry, AIP Conf. Proc. 1345, Wollongong (2011) pp. 75-99; Devic, S. et al. Med. Phys. (2005) 32:2245-2253]. Proper handling of film can reduce noise from dust or debris. Other sources of noise can be mitigated by the application of median or Wiener filters. [Id., citing Lim, J S. Two-Dimensional Signal and Image Processing, Englewood Cliffs, NJ: Prentice Hall (1990)]. Noise from scanner fluctuations can be reduced by acquiring multiple warmup scans before the actual scan is acquired and by averaging the signal from several scans together. [Id., citing Devic, S. et al. Med. Phys. (2005) 32:2245-2253; Devic, S. et al. Med. Phys. (2006) 33:3993-3996; Ferreira, B C et al. Phys. Med. Biol. (2009) 54:1073-1085; Vera Sanchez, J A et al. Phys. Medica. (2016) 32:1167-1174]. Reducing the scan resolution will in turn reduce measured noise through pixel averaging. [Id., citing Williams, M. et al. 4 th SSD Summer School: Concepts and Trends in Medical Radiation Dosimetry, AIP Conf. Proc. 1345, Wollongong (2011) pp. 75-99].

Dose—response and calibration curve. To convert the response of an irradiated RCF into absolute dose, a dose—response or calibration curve is required. It should be emphasized that such calibration curves will be different for each film model [Id., citing Khachonkham, S. et al. PHys. Med. Biol. (2018) 63:065007. 1-11; Massillon-JL, G. et al. Int. J. Med. Phys. Clin. Eng. Radiat. Oncol. (2017) 6:80-92], film batch [Id., citing Mizuno, H. et al. J. Appl. Clin. Med. Phys. (2012) 13:198-205], and readout system [Id., citing Chen, S N et al. Rev. Sci. Instrum. (2016) 87 (073301: pp. 1-6)] for which RCF dosimetry is used. For the same film model, radiation modality, and energy the dose—response curves vary with the particular readout device as well as the color channel (RGB) used in the readout process. [Id., citing Devic, S. et al. Med. Phys. (2007) 34:112-118]. The measured response of the same films scanned on separate scanners of the same model may also be different. Therefore, the application of a calibration curve obtained using one scanner would not be applicable to another even if it is of the same make and model. [Id., citing Soares, C G et al. Clinical Dosimetry for Radiotherapy: AAPM Summer School, Medical Physica Publishing, Madison, WI, ISBN 9781888340846 (2009) Chap. 23, pp. 759-813; Devic, S. Phys. Med. (2011) 27:122-134; Devic, S. et al. Med. Phys. (2007) 34:112-118; Aydarous, A. et al. Results Phys. (2016) 6:952-956].

Dose conversion. Once the relationship between absorbed dose and film response is determined, the calibration function provides the information necessary for the conversion of film response to absorbed dose in subsequently irradiated films. In general, the calibration function is not linear Determining an unknown dose corresponds to finding a root of the calibration function. Many fitting programs used within user-generated codes can create tables of corresponding dose and net OD or PV values, and any proprietary software would convert response to absorbed dose. Alternatively, polynomials [Id., citing Dreindl, R. et al. Z. Med. Phys. (2014) 24:153-163; Borca, V C et al. J. Appl. Clin. Med. Phys. (2013) 14:158-171], power functions [Id., citing Devic, S. et al. Phys. Med. (2016) 32:541-556], rational functions [Id., citing Id.; Micke, A. Med. Phys. (2011) 38:2523-2534], exponential functions [Id., citing Devic, S. et al. Phys. Med. (2016) 32:541-556; Poppinga, D. et al. Med. Phys. (2014) 41:021707.021707-1-021707-8], or other [Id. citing Devic, S. et al. Phys. Med. (2016) 32:541-556; Chiu-Tsao, S T et al. Appl. Radiat. Isot. (2014) 92:102-114; Tamponi, M. et al. Med. Phys. (2016) 43:4435-4446] empirical fitting function can be generated to calculate the dose value corresponding to a netOD or PV in the measurement film.

Correction methodologies. Some correction methodologies may correct or reduce issues related to film non-uniformity, LRA, or improve the dosimetric accuracy of RCF.

Film non-uniformity correction. Variations in the thickness of the active layer of RCFs will result in dosimetric errors if ignored. [Id., citing Mizuno, H. et al. J. Appl. Clin. Med. Phys. (2012) 13:198-205; Hartmann, B. et al. Med. Phys. (2010) 37:1753-1756]. There are several methods to lessen the effects of film non-uniformity. The earliest approach was the use of a double exposure method where a known dose was delivered and the response of the film was measured. [Id., citing Niroomand-Rad, A. et al. Med. Phys. (1998) 25:2093-2115]. An unknown dose was then delivered to the same film and the response corrected for changes in relative response throughout the film. Improvements in the composition of RCFs have led to increased uniformity and double exposure methods have generally been replaced by the dual or triple-channel methods described below. [Id., citing Fuss, M. et al. Phys. Med. Biol. (2007) 52:4211-4225].

Corrections for Color Flatbed Scanners

Single-channel method. Use of a single color channel provides the most basic approach for RCF dosimetry. However, when a single color channel is used any measured signal is directly converted to a dose—response. Artifacts such as film non-uniformity or dust, which disturb the response but are not related to the actual delivered dose, may translate to dosimetric errors. Furthermore, the single-channel method is generally not able to provide any feedback as to the source of the dosimetric errors. [Id., citing Micke, A. et al. Med. Phys. (2011) 38:2523-2534].

Dual-channel method. A great advantage of color flatbed scanners with RGB capability is their ability to measure absorbance values in the red, green, and blue spectral bands. These signals are measured simultaneously and are inherently registered. Use of color scanners allows extension of the dynamic range of RCF to higher doses and the use of multiple color channels can increase dosimetric accuracy by allowing for the removal of dose-independent artifacts.

The active components in RCFs. HD-V2, MD-V3, EBT2, EBT3, and EBT-XD films contain a yellow marker dye that is uniformly incorporated within the active layer. This dye provides high absorbance in the blue color channel in contrast with the active component, which makes a relatively small contribution to net blue optical density. [Id., citing Richley, L. et al. Phys. Med. Biol. (2010) 55:2601-2617]. While there is a change in absorbance in all channels owing to dose, the overall signal in the blue channel is dominated by sensitivity to the thickness of the active layer. In contrast, the overall signals in the red and green channels are more dependent on dose as opposed to differences in thickness. Using a combination of signals from either the red or green color channel with the blue channel provides two measurements of absorbance at each point. This leads to the possibility of accounting for the two variables, dose and thickness, that affect a measured absorbance value, and to compensating for the effects of small thickness non-uniformities in RCF films and thereby improving the reliability of dose measurement. [Id., citing McCaw, T J et al. Med. Phys. (2011) 38:5771-5777].

Triple-channel method. Triple-channel methods use the dose—response from calibrations in all three RGB color channels for calculating dose. [Id., citing Micke, A. et al. Med. Phys. (2011) 38:2523-2534; Mayer, R R et al. Med. Phys. (2012) 39:2147-2155; Azorin, J F P et al. Med Phys. (2014) 41:062101-1-062101-10]. While the dual-channel method is only able to mitigate uncertainties caused by variations in the thickness of the active film layer, triple-channel dosimetry aims to improve upon this by separating the dose-dependent contributions to the film response from any non-dose dependent disturbance which may result not only from variations in film thickness, but from artifacts or noise within the readout system as well. Since the absorbed dose to the film must be independent of color channel, triple-channel methods seek to find the disturbance value, which minimizes the difference between doses from individual color channels. Once the disturbance is found, by separating and removing dose-independent responses from the result, noise in the dose map will be reduced. Several authors have reported RCF dosimetry is improved using triple-channel methods as compared with the single-channel method. [Id., citing Palmer, A L et al. J. Appl. Clin. Med. Phys. (2014) 15:280-296; van Hoof, S J et al. Phys. Med. Biol. (2012) 57:4353-4368; Hayashi, N. et al. J. Radiat. Res. (2012) 53:930-935].

LRA correction. When color flatbed scanners are used to digitize RCF, the response of a uniformly irradiated film may vary with distance from the scanner center along an axis parallel to the light source. For relatively low doses (<1 Gy) when RCFs are positioned within 5-7 cm of the scanner center, the LRA correction is small (<1%). However, the magnitude of the response artifact increases with increasing dose (>1 Gy) and with increasing distance from the lateral center of the scanner. Without LRA correction, dosimetric errors of >30% could occur close to the lateral edges of the scanner and >10% midway between the center and the edge. [Id., citing Lewis, D. Med. Phys. (2015) 42:416-429].

Depending on the application and desired accuracy, it may be necessary to correct for LRA. Several authors have published methodologies to successfully correct for the LRA. [Id., citing Lewis, D F et al. Med. Phys. (2016) 43:643-649; Lewis, D. Med. Phys. (2015) 42:416-429; Saur, S. et al. Med. Phys. (2008) 35:3094-3101; Poppinga, D. et al. Med Phys. (2014) 41:021707.021707-1-021707-8; Paelinck, L. et al. Phys. Med. Biol. (2007) 52:231-242; Menegotti, L. et al. Med. Phys. (2008) 35:3078-3085; Crijns, W. et al. Med Phys. (2013) 40:012102]. Most of these methods generally use a number of films irradiated to different doses and scanned at different lateral positions on the scanner. A correction can then be defined which will adjust the OD [Id., citing Poppinga, D. et al. Med. Phys. (2014) 41:021707.021707-1-021707-8; Paelinck, L. et al. Phys. Med. Biol. (2007) 52:231-242] or PV [citing Lewis, D. et al. Med. Phys. (2015) 42:416-429; Saur, S. et al. Med. Phys. (2008) 35:3094-3101; Menegotti, L. et al. Med. Phys. (2008) 35:3078-3085] measured at positions lateral to the scanner center such that it matches the correct value that would be obtained at the scanner center. Only red channel LRA corrections are in the four studies. [Id., citing Saur, S. et al. Med. Phys. (2008) 35:3094-3101; Poppinga, D. et al. Med Phys. (2014) 41:021707.021707-1-021707-8; Paelinck, L. et al. Phys. Med. Biol. (2007) 52:231-242; Menegotti, L. et al. Med. Phys. (2008) 35:3078-3085]. In contrast, the studies by Lewis and Chan report the LRA correction for all three color channels, red, green, and blue. [Id., citing Lewis, DF et al. Med. Phys. (2016) 61:5426-5442; Lewis, D. Med. Phys. (2015) 42:416-429]. They found that the LRA correction factors are scanner-dependent and color channel dependent. [Id., citing Lewis, D. Med. Phys. (2015) 42:416-429]. Hence, each scanner needs to be commissioned to determine the LRA correction factors unique to it in order to achieve the same level of dose accuracy for large fields.

Recalibration methods. When the “one-scan” protocol is followed [Id., citing Lewis, D. et al. Med. Phys. (2012) 39:6339-6350], an embedded recalibration method [Id., citing Id.; Ruiz-Morales, C. et al. Phys. Medica. (2017) 42:67-75] for further dose correction is used to mitigate inter-scan variability, environmental condition variability and varying post-irradiation OD growth. Therefore, it is necessary to use a recalibration method for further dose correction when the “one-scan” protocol is followed [Id., citing Lewis, D. et al. Med. Phys. (2012) 39:6339-6350]. Several different recalibration algorithms proposed by Lewis et al. and Ruiz-Morales et al. [Id., citing Ruiz-Morales, C. et al. Phys. Medica. (2017) 42:67-75] exist that have been applied to clinical cases.

Gamma Index.

The term “gamma index” or “γ” is a metric for the verification of complex radiotherapy deliveries such as IMTR and volumetric modulated arc radiotherapy (VMAT). The gamma (γ) index introduced by Low et al. Med. Phys. (1998) 25 (5): 656-61 is a quantitative method of comparing two dose distributions and is routinely used for quality assurance (QA) of intensity—modulated radiation therapy (IMRT) treatments. Typically, a two-dimensional measured dose distribution is compared with the planar dose calculated by a treatment planning system (TPS). Performing dose comparisons using the gamma index involves the choice of the dose difference criterion, the distance to agreement (DTA) criterion, and the designation of the reference distribution (either the measured or calculated dose distribution). For each point in the reference distribution, the gamma index is calculated by comparing this point to all points in the evaluated dose distribution within a given search radius, and the gamma index is calculated based on the point in the evaluated distribution that best satisfies both the dose difference and DTA criterion.

The dose and displacement scales were renormalized to be unitless by dividing them by the dose (ΔD) and DTA (Δd) criteria. The displacement between two points, \vec{r}ᵣ and \vec{r}ₑ in the reference and evaluated distributions, respectively, in the renormalized space was termed γ (gamma). The displacement between the noted points can be calculated by Equation 1:

$$Γ ( {\vec{r}}_{e} , {\vec{r}}_{r} ) = \sqrt{\frac{{r}^{2} ( {\vec{r}}_{e} , {\vec{r}}_{r} )}{Δ {d}^{2}} + \frac{{δ}^{2} ( {\vec{r}}_{e} , {\vec{r}}_{r} )}{Δ {D}^{2}}} \qquad ( 1 )$$

where r(\vec{r}ₑ, \vec{r}ᵣ) is the distance between the reference and evaluated points, and δ (\vec{r}ₑ, \vec{r}ₑ is the dose difference. The minimum displacement was be defined as γ (gamma) and calculated by Equation 2:

γ(\vec{r}ᵣ)={Γ(\vec{r}ᵣ,\vec{r}ᵣ)∀{\vec{r}ₑ}} 2)

a gamma (γ) value 1 indicates that the point has passed the acceptance criteria, whereas a gamma value >1 indicates a failing point. Typically, the percentage of points that have passing gamma values determines the overall results of IMRT QA (i.e., whether a particular treatment plan has passed or failed). For instance, a common acceptance criterion is that at least 90% of points need to pass 3%/3 mm criteria for a plan to be considered passing. [Huang, J Y et al. J. Appl. Clin. Med. Phys. (2014) 15 (40: 93-104)].

The gamma index defines for every point in a test dose distribution {D_{R}(x)} a distance measure from a reference dose distribution {D_{T}(y)}, which can be calculated by Equation 3:

$${γ}_{{D}_{T} , {D}_{R}} ( y ) = {min}_{x} \sqrt{{( \frac{y - x}{δ} )}^{2} + {( \frac{{D}_{T} ( y ) - {D}_{R} ( x )}{Δ} )}^{2}} \qquad ( 3 )$$

where δ and A are normalization factors for positions and dose respectively. The convention is to define the normalization factors using the acceptance, e.g., δ=3 mm and Δ=3% of the maximal dose. Then dose quality is considered acceptable if γ_{D}_{_{T}}_{,D}_{_{R}}(y)<1. [Huang, J Y et al. J. Appl. Clin. Med. Phys. (2014) 15 (40: 93-104)].

The term “spatial interpolation” as used herein refers to a process of using points with known values to estimate values at other unknown points. When the dose spacing and DTA criteria are similar, the calculated error in γ in steep dose gradients is large if no interpolation is used. Assuming the dose difference is 0 in the γ calculation, the maximum error in γ is roughly the ratio of the spacing to the DTA criterion, so for example, γ will be accurate to within 0.33 in a steep dose gradient region if the spacing resolution ratio is 3:1. Therefore, a good rule of thumb is that the resolution of the evaluated dose should be no greater than ⅓ the DTA criterion. Interpolation can be used to satisfy this rule.

The problem with spatial resolution had been assumed to be significant only in the case of step dose gradients. However, when the evaluated dose distribution has a low-dose gradient region, similar errors can occur if the location of one of the evaluated points does not coincide with the reference point. Interpolation can reduce these errors, but decrease the y calculation speed performance. [Moften, M. et al. Tolerance limits and methodologies for IMRT measurement-based verification QA: recommendations of AAPM Task Group NO. 218.” Medical Physics (2018) 45 (4): e53-e83] Ju et al [Id., citing Ju, T. et al. Med. Phys. (2008) 35: 879-87] described an algorithm that reduced the interpolation to a geometric problem. They proposed that each evaluated dose distribution line, surface, or hypersurface (for 1D, 2D and 3D dose distributions) be subdivided into simplexes, namely the representations of line or surface elements with the smallest possible number of vertices. These constituted line segments, triangles and tetrahedral. When this subdivision was completed, the closest distance between the reference point and each simplex could easily be computed using only matrix inversion. This sped up the process of calculating y considerably as well as caused each calculation to be implicitly linearly interpolated, removing the errors caused by coarse evaluated dose distribution spacing.

Dose Difference Test. The dose difference at location (r) is the numerical difference 6 between the evaluated dose (Dₑ(\vec{r})) and the reference dose (Dᵣ(\vec{r})) at that location. The dose difference can be calculated by Equation 4:

δ(\vec{r})=Dₑ(\vec{r})−Dᵣ(\vec{r}) (4)

Note that the doses are sampled at the same positions; spatial interpolation is required when they do not. The dose difference criterion typically is described as the percentage of the maximum dose for one or both of the dose distributions being compared (global normalization) or the percentage of the prescription dose. It also can be described as a local dose percentage (local normalization). In global normalization, the dose difference between any measured and calculated dose point pair is normalized using the same value for all point pairs, often the maximum planned dose point. On the other hand, in local normalization, the dose difference for all point pairs is normalized to the planned dose at the local point. Although selecting the local normalization allows one to have the same relative tolerances in the target and critical structure volumes, it will also cause the low-dose regions to have unrealistic dose accuracy requirements.

Computing Systems, Networks and Devices for Implementing Some Embodiments

FIG. 21 schematically depicts a network 300, alternately described as a networked computing system, for implementing some aspects in accordance with some embodiments. Network 300 may include at least one computing system 305, at least one client device 315, and data storage 310 that may be in the form of one or more databases. In some embodiments, network 300 may also include a readout device 317 (e.g., a scanner). In some embodiments, computing system 305, client device 315, readout device 317, and/or data storage 310 may be connected to network 320. However, in other embodiments, two or more of computing system 305, client device 315, readout device 317, and/or data storage 310 may be connected directly with each other, without network 320. While one computing system 305, one client device 315, one readout device 317, and one data storage 310 are shown in FIG. 21, it should be appreciated that any number of computing systems, client devices, readout devices, and data storages could be used.

Computing system 305 may include one or more computing devices configured to perform one or more operations consistent with disclosed embodiments. Computing system 305 is further described in connection with FIG. 22. In some embodiments, computing system 305 may perform at least some aspects or steps of the described methods. In some embodiments, computing system 305, readout device 317, and/or client device 315 may perform at least some aspects or steps of the described methods in some embodiments. For example, in some embodiments, the readout device 317 is employed for obtaining an image of the film and digitizing it. In some embodiments, computing system 305 programmed to perform calibrations and corrections to digitized images obtained from films. In some embodiments, computing system 305 is programmed to obtain information regarding or representing a dose plan. In some embodiments, computing system 305 is programmed to determine or generate a calibration dose plan, which may be based, in part, on a treatment dose plan. In some embodiments, computing system is programmed to generate a patient-specific calibration dose plan based, at least in part, on a patient-specific treatment dose plan.

Data storage 305 may include one or more computing devices configured with appropriate software to perform operations consistent with storing and providing data. Data storage 305 may include, for example, Oracle™ databases, Sybase™ databases, or other relational databases or non-relational databases, such as Hadoop™ sequence files, HBase™, or Cassandra™. Data storage 305 may include computing components (e.g., database management system, database server, etc.) configured to receive and process requests for data stored in memory devices of data storage 305 and to provide data from data storage 305. In some embodiments, data storage 305 may be configured to store digitized image data, or treatment plan, calibration curve coefficients or any other data required by or produced by computing system 305. In some embodiments, data storage 305 or a computing device may be configured to store instructions for implementing a programming and numeric computing platform (e.g., MATLAB from MathWorks, Inc.). In some embodiments, a programming and numeric computing platform may be provided remotely by a server (e.g., via a browser interface). While data storage 305 is shown separately, in some embodiments, data storage 305 may be included in or otherwise related to computing system 305 and/or client device 315.

Client device 315 may include a desktop computer, a laptop, a server, a mobile device (e.g., tablet, smart phone, etc.), a wearable computing device, or other type of computing device. Client device 315 may include one or more processors configured to execute software instructions stored in memory, such as memory included in client device 315. In some embodiments, client device 315 may include software that when executed by a processor performs known Internet-related communication and content display processes. For instance, client device 315 may execute browser software that generates and displays interfaces including content on a display device included in, or connected to, client device 315. Client device 315 may execute applications that allows client device 315 to communicate with components over network 170 and generate and display content in interfaces via display devices included in client device 315. For example, client device 315 may display results produced by computing system 305, such as graphs or images comparing aspects of planned dose distributions and measured dose distributions, polar coordinate plots of isodose curves, etc. Computing system 305 may communicate the results to the client device 315.

Computing system 305, client device 315, and database 315 are shown as a different components. However, computing system 305, client device 315, and/or database 315 may be implemented in the same computing system or device. For example, computing system 305, client device 315, and/or database 315 may be embodied in a single computing device.

Network 320 may be any type of network configured to provide communications between components of network 320. For example, network 320 may be any type of network (including infrastructure) that provides communications, exchanges information, and/or facilitates the exchange of information, such as the Internet, a Local Area Network, near field communication (NFC), optical code scanner, or other suitable connection(s) that enables the sending and receiving of information between the components of network 320. In other embodiments, one or more components of network 320 may communicate directly through a dedicated communication link(s).

FIG. 22 schematically depicts a computing system 400 for implementing some aspects in accordance with some embodiments. In some embodiments, computing device 400 may be computing system 305 shown in FIG. 21. In some embodiments, computing device 400 may be client device 315 shown in FIG. 21. Computing device 400 includes one or more non-transitory computer-readable media for storing one or more computer-executable instructions or software for implementing exemplary embodiments. The non-transitory computer-readable media can include, but are not limited to, one or more types of hardware memory, non-transitory tangible media (for example, one or more magnetic storage disks, one or more optical disks, one or more USB flash drives), and the like. For example, memory 406 included in the computing device 400 can store computer-readable and computer-executable instructions or software for implementing exemplary embodiments. Computing device 400 also includes processor 402 and associated core 404, and optionally, one or more additional processor(s) 402′ and associated core(s) 404′ (for example, in the case of computer systems having multiple processors/cores), for executing computer-readable and computer-executable instructions or software stored in the memory 406 and other programs for controlling system hardware. Processor 402 and processor(s) 402′ can each be a single core processor or multiple core (404 and 404′) processor.

Virtualization can be employed in computing device 400 so that infrastructure and resources in the computing device can be shared dynamically. A virtual machine 414 can be provided to handle a process running on multiple processors so that the process appears to be using only one computing resource rather than multiple computing resources. Multiple virtual machines can also be used with one processor.

Memory 406 can include a computer system memory or random-access memory, such as DRAM, SRAM, EDO RAM, and the like. Memory 406 can include other types of memory as well, or combinations thereof. An individual can interact with the computing device 400 through a visual display device/graphical user interface (GUI) 418, such as a touch screen display or computer monitor, which can display one or more user interfaces 422 for displaying data to the individual. The visual display device 418 can also display other aspects, elements and/or information or data associated with exemplary embodiments. The computing device 400 can include other input devices and I/O devices for receiving input from an individual, for example, a keyboard, a scanner, or another suitable multi-point touch interface 408, a pointing device 410 (e.g., a pen, stylus, mouse, or trackpad). The keyboard 408 and the pointing device 410 can be coupled to the visual display device 418. The computing device 400 can include other suitable conventional I/O peripherals.

The computing device 400 can also include one or more storage devices 424, such as a hard-drive, CD-ROM, or other computer readable media, for storing data and computer-readable instructions and/or software that implements exemplary embodiments of the system as described herein, or portions thereof. Exemplary storage device 424 can also store one or more databases for storing suitable information required to implement exemplary embodiments. The databases can be updated by an individual or automatically at a suitable time to add, delete or update data in the databases. Exemplary storage device 424 can store datasets 426, software 428, and other data/information used to implement exemplary embodiments of the systems and methods described herein. In some embodiments, the storage includes instructions for generating a patient-specific calibration curve based on a planned calibration dose distribution and a measured calibration dose distribution. In some embodiments, the storage includes instructions for generating a gamma index for a specified region, which may be a user-specified region. In some embodiments, the storage includes instructions for computing a gamma index for a specified geometrical region. In some embodiments, the storage includes instructions for determining a radial Hausdorff Distand (rHD) for a specified isodose level for a specific angular orientation. In some embodiments, the storage includes instructions for generating a polar coordinate plot including a planned distribution for a specified isodose level, the measured distribution for the specified isodose level and the rHD for each angle.

The computing device 400 can include a network interface 412 configured to interface via one or more network devices 420 with one or more networks, for example, Local Area Network (LAN), Wide Area Network (WAN) or the Internet through a variety of connections including, but not limited to, standard telephone lines, LAN or WAN links (for example, 802.11, T1, T3, 56kb, X.25), broadband connections (for example, ISDN, Frame Relay, ATM), wireless connections, processing device area network (CAN), or some combination of any or all of the above. The network interface 412 can include a built-in network adapter, network interface card, PCMCIA network card, card bus network adapter, wireless network adapter, USB network adapter, modem or another device suitable for interfacing the computing device 400 to a type of network capable of communication and performing the operations described herein. Moreover, the computing device 400 can be a computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing device 400 can run an operating system 416, such as versions of the Microsoft® Windows® operating systems, the different releases of the Unix and Linux operating systems, a version of the MacOS® for Macintosh computers, an embedded operating system, a real-time operating system, an open source operating system, a proprietary operating system, an operating systems for mobile computing devices, or another operating system capable of running on the computing device and performing the operations described herein. In exemplary embodiments, the operating system 416 can be run in native mode or emulated mode. In an exemplary embodiment, the operating system 416 can be run on one or more cloud machine instances.

### EXAMPLE

The following example is disclosed to provide those of ordinary skill in the art with a complete disclosure and description of how to make and use the present invention, and is not intended to limit the scope of what the inventors regard as their invention nor is it intended to represent that the example and experiments below are all or the only experiments performed. Efforts have been made to ensure accuracy with respect to numbers used (e.g. amounts, dimensions, etc.) but some experimental errors and deviations should be accounted for.

A phantom having the configuration shown in FIGS. 1A-1C and described above was designed and manufactured. The phantom 100 was fabricated from a plastic with an approximate density of 0.93 gm/cm³. The phantom 100 included a body 102 with a peripheral ring portion 115 defining a peripheral film slot 112 and a center cylindrical portion 120 defining a central axis film recess 122. The center cylindrical portion was made from UHMW (Ultra High Molecular Weight) polyethylene. The peripheral ring portion 115 included an inner ring 110 and an outer ring 104. As shown in FIG. 1B, the outer diameter of the outer ring 104 was about 32.0 cm. A diameter of an opening 116 defined by the inner ring 110 was about 24.0 cm. A radial width of the outer ring 104 (as measured between the outer surface of the outer ring 104 and the space 112) was about 1.4 cm. The diameter of the center cylindrical portion 119 was about 10.2 cm. The axial film recess 122 had a square shape about 5.5 cm in length on each side. As shown with respect to FIG. 1C, the axial length of the outer ring 104 (as measured from the front surface 106 to the rear surface 108 in a direction parallel to the central axis 109) was about 15.0 cm; the axial thickness of the support disk 117 (as measured between the front surface 118 of the support disk 117 and the rear surface 108 of the body in a direction parallel to the central axis 109) was about 2.2 cm. The diameter of the center cylindrical portion was about 10 cm; and the axial length from the rear surface 108 to the center axial film recess 122 was about 7.5 cm. In some embodiments, the axial length of the cap 121 (as measured in a direction parallel to the central axis 109) was about 2 cm.

Radiation was detected using gafchromic film positioned in the peripheral ring slot 112 of the peripheral ring portion between the inner ring 110 and the outer ring, and positioned in the center axis film recess 122 of the center cylindrical portion 122 between the base 119 and the cap 121. Specifically, EBT3 gafchromic film made by Ashland LLC was used, however other films could have been employed.

After exposure of the phantom and film to radiation, the peripheral film was removed from the peripheral ring slot and the central axis film was removed from the central axis film recess, and each film was scanned with a flatbed color scanner, specifically an Epson Expression 10000XL scanner can be used; however, alternative color scanners could have been used. Data analysis of the scanned film was performed using methods programmed in the MATLAB programming environment and employed the Imaging Toolbox from MathWorks.

The gafchromic film was in sections of about 10 cm×25 cm, and was measured using the red channel only in the center of a color scanner, e.g., an Epson 10000XL scanner. Another color channel or multiple color channels could have been employed. The scanner was be capable of scanning at a quality of 50 dpi. FIG. 2 is a perspective view of a scanner 150 and a film 152 positioned on the scanner for the scanning phase of the process.

Computerized tomography images of the phantom were obtained using 1 mm slices. Treatment plan axial doses computed with 1 mm resolution were exported as 33×33 cm/500×500 pixels for the periphery and 10×10 cm/200×200 pixels for the center for comparison with digitized information from the films.

Two treatment plans were created to determine the scanner and phantom uniformity. An 8×20 cm 6 MV beam was exposed at 1.5 cm depth and the calculated plan was compared a scanner measurement of an exposed film where the film was at the center of the field in a solid water phantom. FIG. 3 is the uniformity correction plan. FIG. 4 is a top view of a film exposed to a radiation distribution in the solid water phantom according to the calculated plan. The programmed method for evaluating scanner uniformity is described below. The method includes loading or employing a dose to density calibration curve. How the dose to density calibration curve was determined is explained below with respect to FIGS. 7-10. The uniformity plan dose was read for the central plane corresponding to the central axis film and converted to a density. The red channel image data was read for the central axis film. FIG. 5A is a graph of a plan dose converted to density (162) and measured film density (160) for corresponding profiles with agreement within 2%. The ratio of film density to plan density was determined for corresponding profiles and a best linear fit to the profile ratio was determined. FIG. 5B is a graph of a ratio of film density (166) to plan density (168) versus distance for corresponding profiles with a linear fit having a slope of 0.0004 indicating uniform scanner response in a longitudinal direction for the scan region used. If the linear fit had a substantial slope, that slope could have been saved for performing longitudinal laterality correction (LTA) correction on future data.

A phantom plan consisting of multiple static fields with a common isocenter at the phantom origin point (shown in FIG. 6) was created to evaluate the uniform phantom geometry, isocenter location, and phantom density and to show that the calculated plan dose could be in error due to the small gaps between the base and cap. This problem was resolved by assigning a uniform density to the entire phantom.

Converting measured film density to delivered dose was accomplished by creating a density to dose calibration curve. FIG. 7 is an example of a calibration plan used in the phantom with the actual clinical setup to determine a density to dose calibration curve. FIG. 8 is an image of a peripheral ring film exposed to the calibration plan radiation of FIG. 7. In many conventional methods, a few select dose points (e.g., 4 to 6 points) are commonly used to establish a density to dose relationship over a wide range of doses. However, in the method employed in this example, a large number of points for calibration were generated from generated calibration plan that can be tailored based on dose ranges of interest for a patient-specific treatment plan. After a calibration curve was determined, the same calibration curve was used for films specified by the manufacturer to be from the same lot number. The calibration plan for the phantom was created with the same beam parameters (e.g., same energy beam, same depth and with same filters) used for the patient-specific treatment plan.

A fourth-degree polynomial was fit to the density-dose curve and verified by comparing the computed measured dose from the film to the treatment plan dose extracted from cylindrical verification plan. FIG. 9 is a graph of measured film dose (170) as compared to the plan dose (172), and FIG. 10 is a graph of fourth-degree polynomial order fit (176) to the dose versus intensity calibration curve (174).

A statistically significant number of points were employed to determine the accurate calibration curve for the film in FIG. 10. The method employed by the system to determine the calibration curve included reading the red channel of the peripheral ring calibration film and converting the digitized images to 0.5 mm pixel sizes. A mean profile film measurement was determined from the average of 21 central slices of the film with a slice separation of 0.5 mm . . . The calibration plan dose at a central plane of the peripheral film was analyzed and read.

First, the center and radius of the cylinder plan were determined to correspond to values calculated by the treat planning system. The cylindrical body of the phantom plan was rotated in increments equivalent to 0.5 mm arc length in the peripheral film plane and plan dose was recorded at the peripheral film depth for each rotation. The best fit fourth order polynomial was determined for film density as a function of plan dose. The polynomial coefficients for each fit were electronically stored in the database associated with the system.

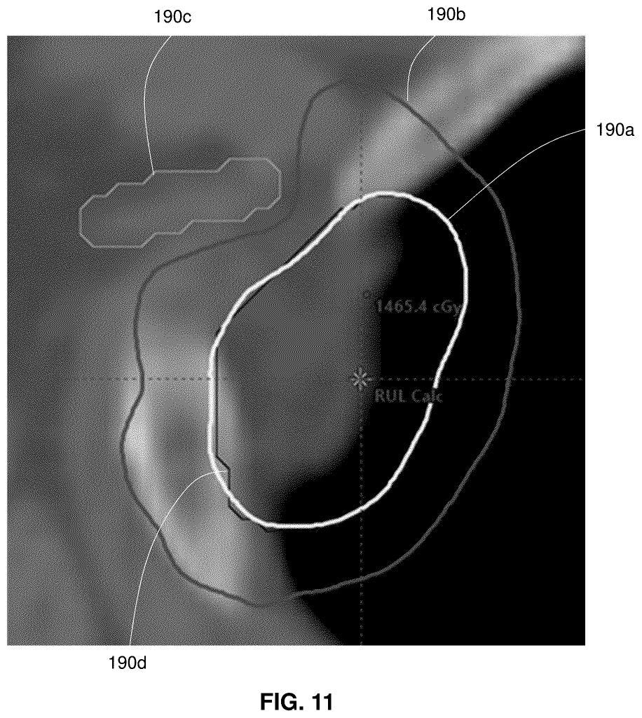

For demonstration purposes a representative volumetric modulated arc radiotherapy (VMAT) SBRT lung patient plan with two 180-degree arcs was created with the planning target volume (PTV) near the brachial plexus. FIG. 11 is a representative SBRT plan generated by the system in which yellow line 190a, and red line 190b are isodose contours, green line 190c indicates the brachial plexus, and the blue line 190d is the target. The plan was for five fractions with a prescribed dose of 1000 cGy per fraction. The red line (190b) represents 640 cGy per fraction equivalent to 3200 cGy total, which is the desired dose limit for the brachial plexus. Similarly, the yellow line (190a) represents 1000 cGy per fraction equivalent to 5000 cGy total which is the total prescribed target dose

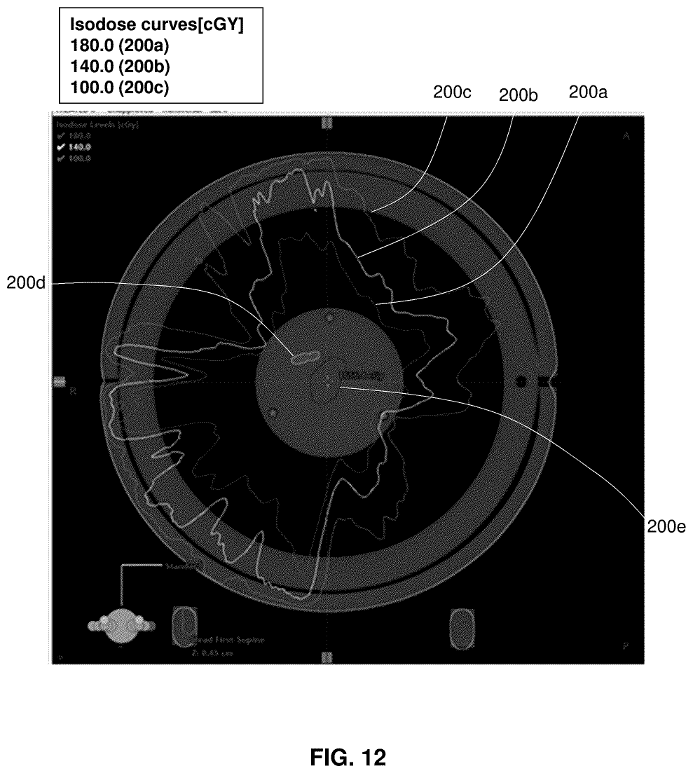

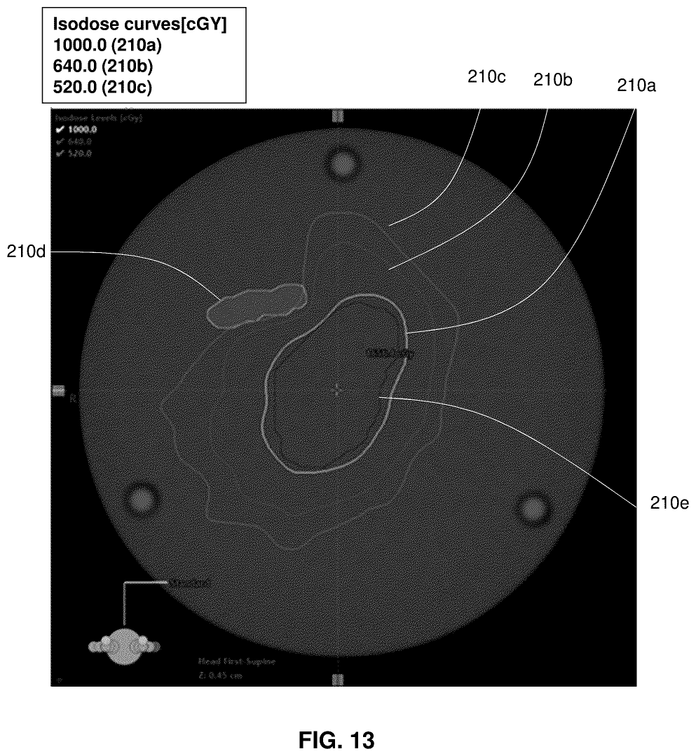

A corresponding verification plan was created on the phantom with doses extracted in the periphery encompassing the entire treatment field and the clinically significant axial plane where the brachial plexus is closest to exceeding tolerance. FIG. 12 is a view of a verification plan including the peripheral region and isodose curves 200a (red) for 180 cGy, 200b (yellow) for 140 cGy, and 200c (purple) for 100 cGy, and lines indicating the brachial plexus 200d (green) and the target 200e (blue). FIG. 13 is view of a central region of the verification plan including isodose curves 210a (yellow) for 1000 cGY, 210b (red) for 640 cGY, and 210c (purple) for 520 cGY and lines indicating the brachial plexus 210d (green) and the target 210e (blue). As an added unique feature, various plan structures such as target and brachial plexus were represented in the axial verification plan so that the analysis could focus on specific geometrical regions.

MATLAB with the imaging toolbox was used for computation. All scans and exported dose planes were converted to 0.5×0.5 mm pixels and registered to corresponding data sets. A global gamma index was calculated using the classical formula with 3% and 2 mm criteria with 10% threshold. The Example also included a method for a customizable gamma index that enabled determination of global and local indices for clinically relevant areas such as the quadrant containing the brachial plexus in the axial plane. For example, conventional methods often provide a single Gamma Index (either global or local) for the entire irradiated volume. One of the major complaints about the gamma index is that it contains no geometrical information. Custom methods were developed to enable the index to be determined in specific geometrical areas. For example, in a 180-degree arc treatment, conventional commercially available cylindrical phantoms commonly include measurements in the clinically irrelevant exit region and artificially skew the results. The custom methods developed enable the analysis to be limited to the entrance portion of the peripheral cylinder for 180-degree treatment arcs, which is desirable because the relatively uniform exit dose distribution is clinically irrelevant and with global gamma index settings, this artificially skews the gamma index to a high passing rate.

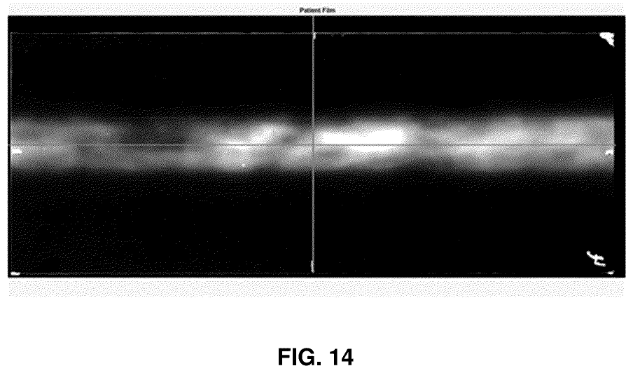

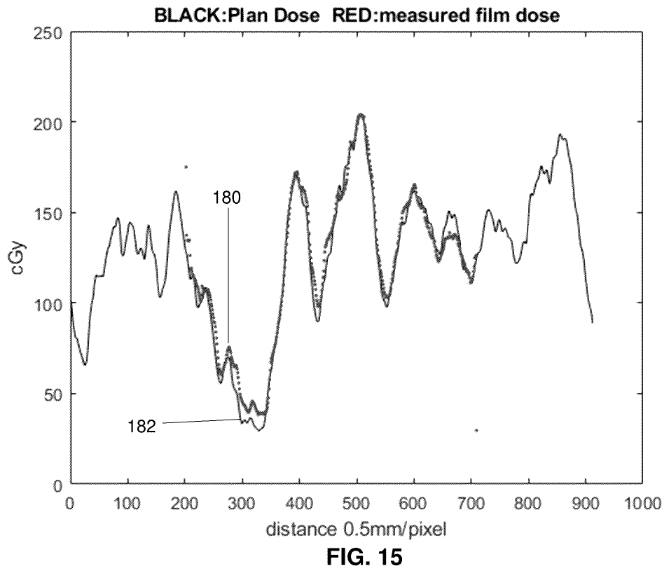

Besides Gamma index, the individual measured versus plan dose profiles in the periphery contained detailed information could be useful to identify specific faults in individual control points, faults at certain gantry angles, or excess failures in high gradient sections. FIG. 14 is a top view of a peripheral film (e.g., a film located between the outer and inner rings of the phantom), and FIG. 15 is a graph of a profile plot with the measured film plan (180) as compared to a plan (182) along a profile.

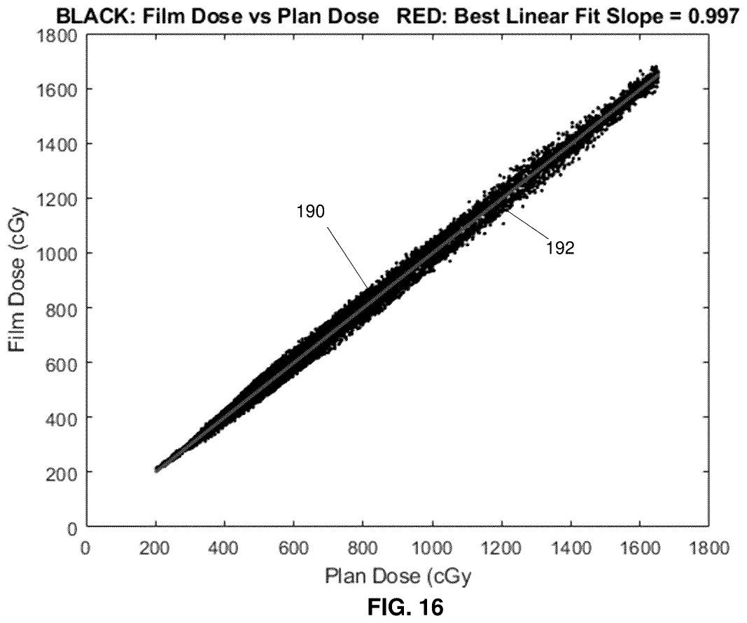

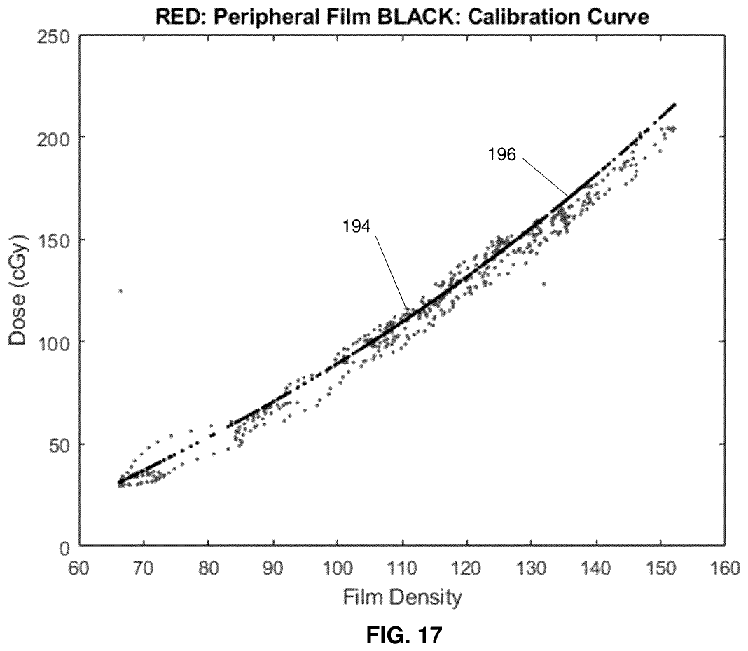

As an internal QA process the registered plan dose was plotted as a function of the corresponding film density superimposed on the known calibration curve to highlight any discrepancy. The measured film dose was plotted as a function of the plan dose to highlight any discrepancy (see FIG. 16). The best least squares linear fit should have a slope of 1.0 and intercept at 0.0 and a minimal standard deviation. FIG. 16 shows a good linear fit with a slope of 0.997 and an intercept close to 0 and small deviations. FIG. 17 is a scatter plot of peripheral film dose (e.g., measured dose) (194) versus film density fit by a fourth order polynomial (196) to determine a calibration curve

Results

Scanner uniformity in the central region encompassing the stated film sections was acceptable without linearity correction as noted in AAPM TG235. Other guidelines were implemented as necessary.

AAPM TG218 recommends that spatial resolution of the evaluated dose should be no greater than ⅓ the DTA criterion, i.e. 0.67 mm for 2 mm DTA. MatLab image size tool was used to convert all planar data to 0.5×0.5 mm pixel size. The 1 mm slice thickness however was not interpolated.

Phantom uniformity initially was unacceptable due to small variations in the film position within the peripheral slot. Acceptable phantom uniformity was achieved by overriding the measured phantom CT density with a uniform density.

Scatter plots used to assure process integrity corroborated the calibration curve. For the center plane the planned versus measured slope and intercept were 0.997 and 1.12.

The gamma index calculation was verified by comparison with a commercial product for various fields and parameters.

Gamma passing rate in the periphery for the sample case was 98.4%. Many failures occurred along two straight lines possibly indicating film scratches or scanner artifacts warranting further investigation. The remaining failures were clustered in two low dose regions.

For the central region the gamma passing rate was 100%. To verify this exceptional result, the plan and measured doses were misregistered and as expected the passing rate decreased proportionally. Vertical and horizontal profiles comparing plan and measured doses corresponded almost exactly even in the high gradient region across the brachial plexus.

Polar coordinate plots of specific isodose lines were valuable in showing the comparison between plan dose and measured film dose in a clinically relevant axial plane. The difference in radial distance between the plan dose and measured film dose for each angle in one-degree increments was similar to DTA in gamma calculations. However, the visual plot demonstrates where the deviations are located relative to a critical organ such as the brachial plexus located at 330-degrees. The maximum of the absolute value of all the radial differences is analogous to the Hausdorff Distance (HD) metric used in topology and is a valuable supplement to the Gamma Index.

Case 1. The film was intentionally misregistered to the plan and resulting in large variations in radial distances exceeding 3 mm. This would be unacceptable in the brachial plexus region and tolerance dose would be exceeded. FIG. 18A is a polar plot of an isodose curve for an intentionally misregistered scanned film (197a, red) and a corresponding isodose curve for the plan (197b, black). FIG. 18B is a radial distance error plot for the isodose curves of FIG. 18A (dots-film, lines-plan).

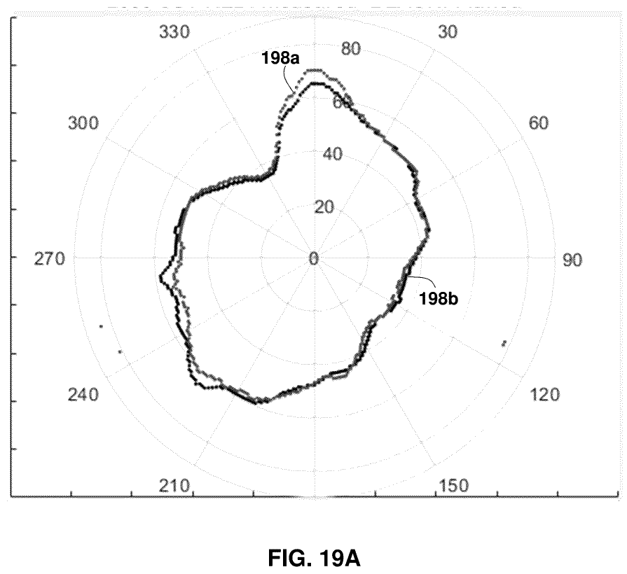

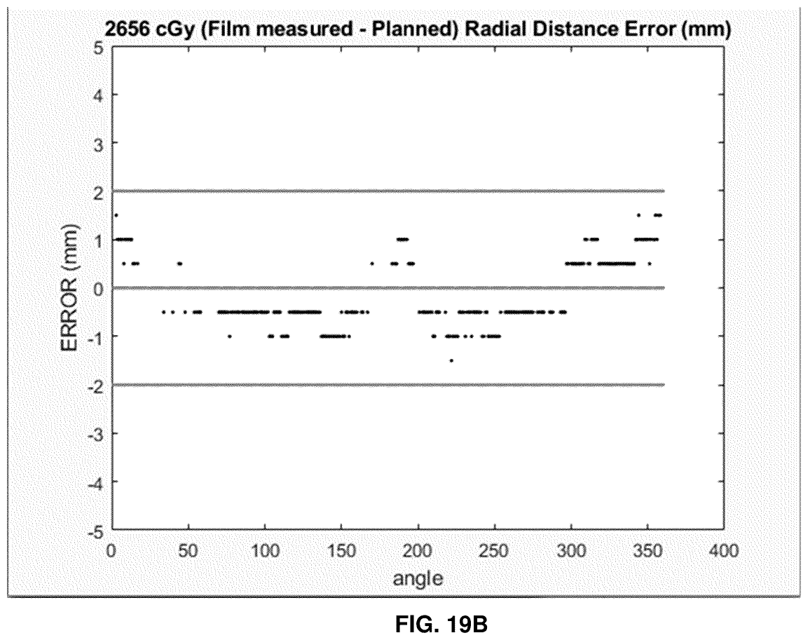

Case 2. With the correct registration, the results within 1.5 mm error were acceptable and the brachial plexus would be within tolerance. FIG. 19A is a polar plot of an isodose curve for the correctly registered scanned film (198a, red) and a corresponding isodose curve for the plan (198b, black). FIG. 18B is a radial distance error plot for the isodose curves of FIG. 19A.

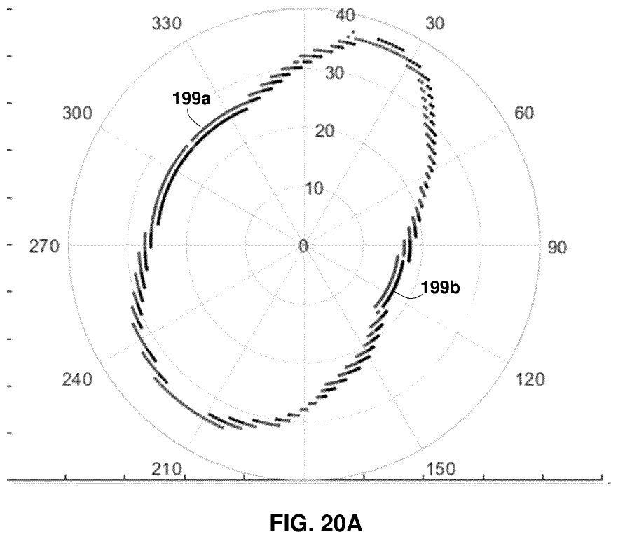

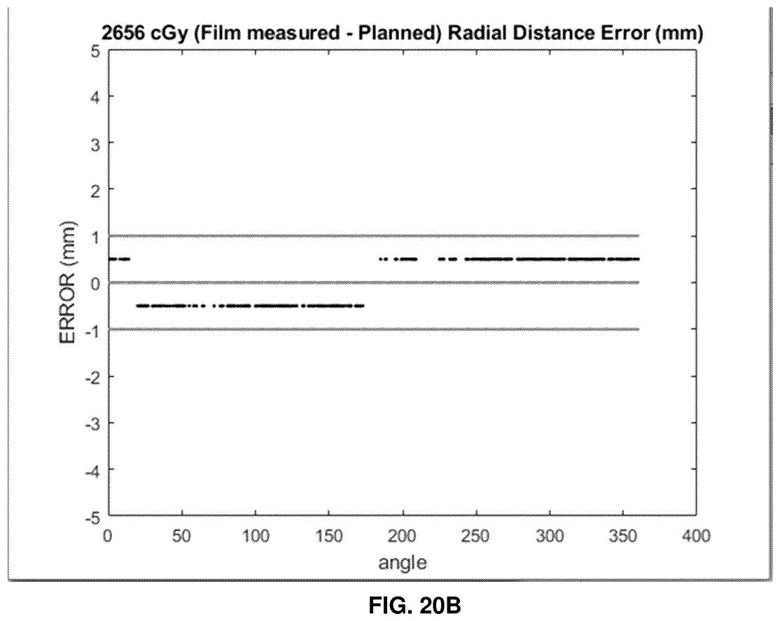

Case 3. For the target dose the error was within the pixel size of 0.5 mm. This measurement exceeded all expectations and demonstrated the power of film as a QA medium. Furthermore the ability of the delivery Linac to exactly replicate the planned dose is clearly demonstrated and shows that the phantom and methods and computer-implemented methods presented are invaluable in end-to-end Linac and treatment planning system commissioning. FIG. 20A is a polar plot of an isodose curve for a scanned film (199a, red) and a corresponding isodose curve for the plan (199b, black). FIG. 20B is a radial distance error plot for the film measured plan of FIG. 20A.

Conclusions

A significant number of IROC credentialing submissions fail even though they have passed in-house QA. The proposed phantom and methods and computer-implemented methods provide simultaneous peripheral and axial data, the ability to focus on specific geometrical regions, superb resolution with statistical validity, and built-in versatility and verification. These innovative factors enabled enhanced performance that warrant the additional effort that may be associated with film dosimetry and custom phantoms.

While the present invention has been described with reference to the specific embodiments thereof, it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A phantom, comprising:
a body including a peripheral ring portion defining a central axis of the body and of the phantom and a center portion at least partially encircled by the peripheral ring portion or fully encircled by the peripheral ring portion, the center portion aligned with and extending along the central axis of the body;
a peripheral film holder defined by the peripheral ring portion and at least partially encircling or fully encircling the central axis, the peripheral film holder configured to receive and hold at least one peripheral film; and
an axial film holder defined by the center portion and oriented perpendicular to the central axis, the axial film holder configured to receive and hold at least one axial film.

2. The phantom of claim 1, wherein the peripheral ring portion fully encircles the center portion.

3. The phantom of claim 2, wherein the peripheral film holder fully encircles the central axis.

4. The phantom of claim 1, wherein the phantom is configured to be exposed to radiation such that both the at least one peripheral film and the at least one second axial film are simultaneously exposed to the radiation.

5. The phantom of claim 1, wherein the peripheral ring portion includes an inner ring and an outer ring at least partially encircling the inner ring, at least a portion of the outer ring removably coupled to or configured to be removably coupled with the inner ring; and
wherein the peripheral film holder is a peripheral slot.

6. The phantom of claim 5, wherein at least a portion of the outer ring is removable from the inner ring to expose at least about 180° of a film supporting surface of the inner ring.

7. The phantom of claim 5, wherein the peripheral slot is formed between a radially inward facing surface of the outer ring and a radially outward facing surface of the inner ring.

8. The phantom of claim 5, wherein the peripheral slot extends at least about 180° about the center axis between the outer ring and the inner ring.

9. The phantom of claim 5, wherein the peripheral slot extends at least about 270° about the center axis between the outer ring and the inner ring.

10. The phantom of claim 5, wherein the peripheral slot extends about 360° about the center axis between the outer ring and the inner ring.

11. The phantom of claim 1, wherein the peripheral film holder is configured such that at least one peripheral film positioned in the peripheral film holder would have a front surface or a back surface facing a cylindrically shaped surface of the peripheral film holder with an axis of a cylinder of the cylindrically shaped surface being the central axis of the phantom.

12. The phantom of claim 1, wherein the center portion includes a base and a cap configured to be removably coupled to the base section.

13. The phantom of claim 12, wherein the axial film holder is defined between the base and the cap.

14. The phantom of claim 12, wherein a surface of the base facing the cap includes a recess configured to receive the at least one axial film.

15. The phantom of claim 1, wherein the axial film holder is oriented perpendicular to the central axis of the body.

16. The phantom of claim 1, wherein the peripheral film holder configured to receive and hold the at least one peripheral film is configured to hold at least one radiochromic film, and wherein the axial film holder configured to receive and hold the at least one axial film is configured to hold at least one radiochromic film.

17. A computer-implemented method for determining a density to dose calibration for a radiochromic film to be used with a phantom for evaluating a radiation therapy plan, the method comprising:
receiving or accessing data corresponding to a digitized image of a first radiochromic film exposed to radiation while positioned in the phantom according to a calibration plan including multiple different levels of radiation along the first radiochromic film and generating two-dimensional film density data corresponding to the received and accessed data for the first radiochromic film;
determining an average film density profile along a direction in the two-dimensional film density data by averaging a plurality of profiles parallel to the direction;
determining a plan dose profile corresponding to the direction in the film based on the calibration plan;
determining a best fit fourth order polynomial and corresponding coefficients for plan dose as a function of film density for the first radiochromic film based on the determined average film density profile and the determined plan dose profile, thereby determining a density to dose calibration for the first radiochromic film; and
applying the best fit fourth order polynomial as a calibration curve for received or accessed data regarding a second exposed radiochromic film having the same film characteristics as those of the first radiochromic film and used with the phantom for evaluating the radiation therapy plan.

18. The method of claim 17, further comprising generating the calibration plan based on one or more characteristics of a patient-specific treatment plan.

19. The method of claim 17, wherein the first radiochromic film was disposed at a peripheral ring of the phantom that encircled a central axis of the phantom during exposure.

20. A computer-implemented method for determining a gamma index for verification or quality assurance of a radiation therapy patient treatment plan, the method including:
receiving or accessing plan data regarding the radiation therapy patient treatment plan;
receiving or accessing exposed film data corresponding to at least one digitized image of at least one radiochromic film exposed to radiation in a cylindrical phantom according to the radiation therapy patient treatment plan;
converting density in the at least one digitized image to film dose to produce at least one two-dimensional film dose map using a calibration curve;
receiving information regarding or identifying a geometric region of interest;
determining film dose values corresponding to the geometric region of interest from the at least one two-dimensional film dose map;
determining plan dose values for the radiation therapy patient treatment plan corresponding to the geometric region of interest; and
generating a gamma index specific to the region of interest based, at least in part, on the determined film dose values and determined plan dose values corresponding to the geometric region of interest.

21. The method of claim 20, wherein the at least one film includes a film that was positioned on a cylindrical surface of the phantom during exposure.

22. The method of claim 20, wherein the at least one film includes a film that was at a center of the phantom and oriented perpendicular to a central axis of the phantom during exposure.

23. The method of claim 20, wherein the at least one film includes at least one peripheral film that was disposed on a cylindrical surface of the phantom during exposure and at least one central film that was disposed at the center of the phantom and was oriented perpendicular to a central axis of the phantom during exposure.

24. The method of claim 20, wherein the region of interest includes an area corresponding to a position of the at least one peripheral film.

25. The method of claim 20, wherein the received information regarding or identifying the geometric region of interest is received from a user.

26. A non-transitory computer readable medium storing instructions that when executed by at least one processor perform the method of claim 20.

27. A computer-implemented method for quality assurance of a radiation therapy patient treatment plan, the method including:
receiving data corresponding to a digitized image of a radiochromic film exposed to radiation in a cylindrical phantom according to the radiation therapy patient treatment plan with the radiochromic film disposed at a central axis of the cylindrical phantom and oriented perpendicular to the cylindrical phantom;
converting density in the digitized radiochromic film image to film dose to produce a two-dimensional film dose map using a calibration curve;
determining a plan dose map corresponding to a plane of the radiochromic film from the radiation therapy patient treatment plan;
receiving or determining an isodose curve for a first isodose value for the film dose map;
receiving or determining an isodose curve for a first isodose value for the plan dose map;
determining a radial distance to the isodose curve for the film dose map and determining a radial distance to the isodose curve for the plan dose map at each of a plurality of orientations; and
displaying radial distances to the isodose curves for the film dose map and for the plan dose map at the plurality of orientations in polar coordinates.
